(19) United States
(12) Patent Application Publication
(10) Pub. No.: US 2004/0110775 A1
(43) Pub. Date: Jun. 10, 2004

(54) Cyclin dependent kinase inhibiting purine derivatives

(76) Inventors: Roger John Griffin, Morpeth (GB); Alan Hilary Calvert, Blaydon Burn (GB); Nicola Jane Curtin, Rowlands Gill (GB); Bernard Thomas Golding, Little Benton (GB); Ian Robert Hardcastle, Hexham (GB); David Richard Newell, Hexham (GB); Philip John Jewsbury, Altrincham (GB); Francis Thomas Boyle, Congleton (GB); Jane Anne Endicott, Oxford (GB); Martin Edward Mantyla Noble, Oxford (GB)

Correspondence Address: PILLSBURY WINTHROP, LLP, P.O. BOX 10500, MCLEAN, VA, 22102, US

(21) Appl. No.: 10/466,693

(22) PCT Filed: Jan. 22, 2002
(86) PCT No.: PCT/GB02/00272

(30) Foreign Application Priority Data
Jan. 23, 2001 (GB) GB 0101686.4

(51) Int. Cl. A61K 31/52; A61K 31/522; C07D 473/10

(52) U.S. Cl. 514/263.2; 514/263.37; 544/276; 544/277

(57) ABSTRACT

The present invention relates to a series of CDK-inhibiting purine derivatives of structural formula (I), or a pharmaceutically acceptable salt and/or prodrug form thereof, wherein: X is O, S or CHR_{X} where R_{X} is H or C₁₋₁₄ alkyl; D is NZ₁Z₂ where Z₁ is selected from H, C₁₋₄ alkyl, C₁₋₄ hydroxyalkyl, an unsubstituted or substituted aryl or heteroaryl, and an unsubstituted or substituted aralkyl or heteroaralkyl group, and Z₂ is selected from an unsubstituted or substituted aryl or heteroaryl, and an unsubstituted or substituted aralkyl or heteroaralkyl group; A is selected from H, C₁₋₄ alkyl, C₁₋₄ alkoxy, hydroxy, CH₂(CH₂)ₙOH (n=1-4), and NRₐ₁Rₐ₂ where Rₐ₁ and Ra₂ are each independently H or C₁₋₄ alkyl; B is selected from H, halo, C₁₋₄ alkyl, C₁₋₄ alkoxy, CF₃, an optionally substituted aryl or an optionally substituted aralkyl, and a hydroxy group that may undergo a C═O tautomeric rearrangement; and Y comprises an unsubstituted or substituted 4- to 8-membered carbocyclic or heterocyclic ring, optionally forming part of a larger fused ring structure, or consists of an optionally substituted linear or branched hydrocarbon chain. These purine derivatives are potential chemotherapeutic agents and, accordingly, the present invention also relates to the use of these compounds in the treatment of tumours or other cell proliferation disorders and pharmaceutical compositions comprising these compounds.

### FIELD OF THE INVENTION

The present invention relates to certain compounds, especially purine derivatives, which show activity in biological systems as cyclin dependent kinase (CDK) inhibitors and which are accordingly of interest as potentially useful therapeutic agents that may be incorporated in pharmaceutical compositions or formulations for use in controlling or inhibiting cell growth or proliferation in mammals, for example in connection with antitumour or cancer treatment.

### BACKGROUND

Cyclin dependent kinases (CDK's) are a family of enzymes which form complexes with other activating proteins known as cyclins to provide key regulatory factors that are involved in the control of growth and division in animal cells. More particularly, the progression of animal cells through the cell division cycle (G1, S, G2 and M phases) is regulated by the sequential formation, activation and subsequent inactivation of a series of CDK/cyclin dimer complexes which control passage past cell cycle checkpoints and transitions between successive phases of the cell cycle, with the CDK's acting as catalytic sub-units of the complexes.

There are in fact a number of different cyclin proteins which, like the different CDK's, form a somewhat loosely related family of CDK-activating proteins. It is also known that the different CDK/cyclin complexes function at different stages of the cell cycle with sequential increase and decrease in cyclin expression during the cell cycle and cyclin degradation during M phase usually being an important factor in determining orderly cell cycle progression. Thus, progression through the G1 to the S phase of the mammalian cell cycle is believed to be regulated primarily by cyclin dependent kinases CDK2, CDK3 and CDK4 (and possibly also CDK6 in some cells) in association with at least cyclins D and E, the complexes of CDK2 and CDK4 (and possibly CDK6) with D type cyclins in particular playing an important role in controlling progression through the G1 restriction point whilst the CDK2/cyclin E complexes are essential for bringing about the transition from G1 into S phase. Once the S phase is entered it is believed that further progression and entry into the G2 phase of the cell cycle requires activated complexes of CDK2 with another cyclin which is designated cyclin A, i.e. complexes CDK2/cyclin A. Finally, for the transition from G2 phase to M phase and the initiation of mitosis, activated complexes of the cyclin dependent kinase designated CDK1 (also known as Cdc2) with a cyclin designated cyclin B (and also complexes of CDK1 with cyclin A) are required.

In general, control of the cell cycle and activity of CDK's involves a series of stimulatory and inhibitory phosphorylation and dephosphorylation reactions, and in exercising their regulatory functions the CDK/cyclin complexes, when activated, use ATP as a substrate to phosphorylate a variety of other substrate cell proteins, usually on serine and threonine groups thereof. Control of the cell cycle may also involve inhibitors of CDK/cyclin complexes which block the catalytic function of these enzymes so as to lead to arrest of the cell cycle. Certain natural inhibitors, such as for example the inhibitory proteins known as p16 and p21, can block cell cycle progression by binding selectively to CDK/cyclin complexes to inactivate the latter.

Control of CDK function by inhibitors may therefore provide a further mechanism for controlling cell cycle progression, and this has led to proposals for using CDK inhibitors as antiproliferative therapeutic agents, in antitumour therapy for example, for targeting abnormally proliferating cells and bringing about an arrest in cell cycle progression. This has seemed to be especially appropriate since it is known that severe disorders or irregularities in cell cycle progression frequently occur in human tumour cells, often accompanied by over-expression of CDK's and other proteins associated therewith. Also, compared with established cytotoxic antitumour drugs, the use of inhibitors of cell proliferation acting through CDK's would have the advantage of avoiding a direct interaction with DNA, thereby giving a reduced risk of secondary tumour development.

The potential therapeutic applications and other possible uses have accordingly led to a search for a wider range of chemical inhibitors of CDK's, especially selective inhibitors that may be suitable for pharmaceutical use. Inhibitory activity and selectivity of selected CDK/cyclin complexes is generally assayed by measuring the kinase activity in phosphorylating the protein histone H1 (one of the major protein constituents of chromatin which generally provides a good CDK substrate) in the presence of the suspected inhibitor under test. A number of compounds having potentially useful CDK inhibitory properties that have been identified in this way are described in a review article, of which the content is incorporated herein by reference, entitled “Chemical inhibitors of cyclin-dependent kinases” by Laurent Meijer published in Cell Biology (Vol. 6), October 1996. Among the compounds referred to in the above-mentioned article is a potent CDK1 and CDK2 inhibiting adenine derivative 2-(2-hydroxyethylamino)-6-benzylamino-9-methyl-purine, named “olomoucine”, and also a close analogue incorporating modifications at positions 2, 6 and 9, namely, 6-(benzylamino)-2(R)-[{1-(hydroxy-methyl)propyl}amino]-9-isopropylpurine. This latter compound is named “roscovitine” and is even more potent than olomoucine as a CDK inhibitor. The strong but selective CDK inhibitory properties of olomoucine were first described in a paper by J. Vesely et al entitled “Inhibition of cyclin-dependent kinases by purine analogues”, Eur. J. Biochem. 224, 771-786 (1994), and further studies on CDK inhibitory properties of a range of purine compounds in the form of adenine derivatives, including olomoucine and roscovitine, are reported and discussed in a paper by L. Havlicek et al entitled “Cytokinin-Derived Cyclin-Dependent Kinase Inhibitors: Synthesis and cdc2 Inhibitory Activity of Olomoucine and Related Compounds” J. Med. Chem. (1997) 40, 408-412. Again, the content of these publications is to be regarded as being incorporated herein by reference.

The inhibitory activity of both olomoucine and roscovitine has been shown to result from these compounds acting as competitive inhibitors for ATP binding. It may be noted that olomoucine at least is reported as having a total lack of inhibitory activity in relation to many common kinases other than CDK's. Selectivity is further manifest by the fact that both olomoucine and roscovitine inhibit activity of CDK1, CDK2 and CDK5, but neither has been found to be active against CDK4 or CDK6.

Olomoucine in particular has been regarded as providing a lead compound for helping to identify and design further purine based CDK inhibitors, and based on structure/activity studies it was suggested in the above-mentioned paper of Vesely et al that N9 substitution by a hydrophobic residue such as methyl, 2-hydroxyethyl or isopropyl was important, e.g. to provide a direct hydrophobic interaction with the CDK, and that a side chain at C2 appeared to be essential. Similarly, in the paper of Havlicek et al, apart from observing that for purine compounds to have CDK inhibitory activity the 1 and 7 positions, and possibly the 3 position, of the purine ring must remain free to permit hydrogen bonding, it was also stated that a polar side chain at position 2 appears to be essential and that N9 substitution by a hydrophobic residue is also probably important for positive binding. Positions 2, 6, 7 and 9 in the purine ring were identified as being the positions which control binding to CDK1.

In the review article of Meijer, it is also mentioned that as a result of crystallization of CDK-inhibitor complexes, and in particular co-crystallization studies with CDK2, it has been found that inhibitors such as olomoucine and roscovitine localize in the ATP binding pocket which is located in the cleft between the small and large lobes of the CDK protein molecule, and that specificity was probably provided by portions of the inhibitor molecules interacting with the kinases outside the ATP binding sites.

Subsequently, however, as disclosed in the published specification No. WO 99/02162 of our PCT International Patent Application No. PCT/GB98/02025 which may be read in conjunction with the present specification, it was found unexpectedly that the compound O⁶-cyclohexylmethylguanine which has no substituents at either the 2-NH₂ position or the 9 position in the purine ring and which has 6-NH replaced by 6-O, was nonetheless cytotoxic and showed very high inhibitory activity, comparable to that of olomoucine, against CDK1(cdc2)/cyclin B complexes.

Also, as disclosed in WO 99/02162 other guanine derivatives have been identified, more closely related to O⁶-cyclohexylmethylguanine than to compounds such as olomoucine and roscovitine, which show significant CDK inhibitory activity, and crystallographic studies have revealed that complexes of CDK2 (homologous with CDK1, at least in respect of the catalytic binding site) with guanine derivatives such as O⁶-cyclohexylmethylguanine and O⁶-cyclohex-1-enylmethylguanine bind together in a different manner from complexes of CDK2 with the adenine derivative olomoucine. This is explained further in WO 99/02162.

### SUMMARY OF THE INVENTION

The purine compounds disclosed in WO 99/02162 which were considered to have CDK-inhibiting activity useful for treating tumours and other cell proliferation disorders in mammals were defined by the following structural formula:

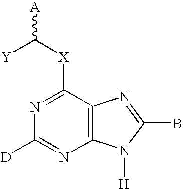

wherein

X is O, S or CHRₓ

where Rₓ is H or C₁₋₄ alkyl;

D is H, halo or NZ₁Z₂

where Z₁ and Z₂ are each independently H or C₁₋₄ alkyl or C₁₋₄ hydroxyalkyl;

A is selected from H, C₁₋₄ alkyl, C₁₋₄ alkoxy, hydroxy, CH₂(CH₂)ₙOH (n=1-4), and NRₐ₁Rₐ₂ where Rₐ₁ and Rₐ₂ are each independently H or C₁₋₄ alkyl;

B is selected from H, C₁₋₄ alkyl, C₁₋₄ alkoxy, CF₃, an optionally substituted aryl (e.g. phenyl) or an optionally substituted aralkyl (e.g. benzyl), and an hydroxy group that provides a C═O tautomer; and

Y is or includes an optionally substituted 4- to 8-membered carbocyclic or heterocyclic ring; or comprises an optionally substituted linear or branched hydrocarbon chain.

For effective CDK inhibiting activity it was believed that when the substituent D at the 2-position of the purine ring is a group NZ₁Z₂ as specified above, it was necessary for Z₁, and Z₂ each to be independently H, C₁₋₄ alkyl or C₁₋₄ hydroxyalkyl. It was further believed that substituent D would usually be NH₂, i.e. Z₁ and Z₂ would each be hydrogen. It has now become apparent, however, that in such purine compounds Z₁ and Z₂ can each independently be an optionally substituted aryl, aralkyl, heteroaryl or heteroaralkyl group and, surprisingly, it has been found that such compounds can have a CDK-inhibiting activity comparable with or superior to the activity of the compounds disclosed in WO 99/02162. Indeed, when Z₁ and Z₂ are aryl or heteroaryl groups provided with selected substituents, especially hydroxyl, hydroxyalkyl, acyl, carboxyl, cyano, and most especially carboxamide (carbamoyl), sulfonamide (sulfamoyl), sulfone, sulfoxide or some other sulfur based substituents, a significant increase in CDK-inhibiting activity is obtained which in some cases, at least with sulfonamide based substituents, can even be considered dramatic.

Thus, in contrast to the disclosure in WO 99/02162 the present invention provides purine compounds having CDK-inhibiting activity useful for treatment of tumours and other cell proliferation disorders in mammals, said compounds having the structural formula I below:

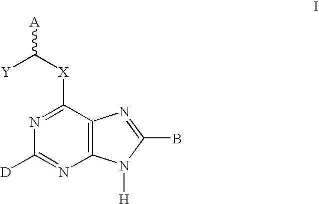

or a pharmaceutically acceptable salt and/or prodrug form thereof, wherein:

X is O, S or CHRₓ

where Rₓ is H or C₁₋₄ alkyl;

D is NZ₁Z₂

where Z₁ is selected from H, C₁₋₄ alkyl, C₁₋₄ hydroxyalkyl, an unsubstituted or substituted aryl or heteroaryl, and an unsubstituted or substituted aralkyl or heteroaralkyl group, and Z₂ is selected from an unsubstituted or substituted aryl or heteroaryl, and an unsubstituted or substituted aralkyl or heteroaralkyl group;

A is selected from H, C₁₋₄ alkyl, C₁₋₄ alkoxy, hydroxy, CH₂(CH₂)ₙOH (n=1-4), and NRₐ₁Rₐ₂ where Rₐ₁ and Rₐ₂ are each independently H or C₁₋₄ alkyl;

B is selected from H, halo, C₁₋₄ ally, C₁₋₄ alkoxy, CF₃, an unsubstituted or substituted aryl or an unsubstituted or substituted aralkyl, and an hydroxy group that may undergo a C═O tautomeric rearrangement; and

Y comprises an unsubstituted or substituted 4- to 8-membered carbocyclic or heterocyclic ring, optionally forming part of a larger fused ring structure, or consists of an optionally substituted linear or branched hydrocarbon chain.

The invention flier includes the CDK-inhibiting purine compounds which are new chemical entities or which are at least new therapeutic agents.

The term “aryl” is used herein to denote a carbocyclic group or structure having at least one aromatic ring that in some cases may form part of a multiple condensed ring structure. Preferably “aryl” substituents, when present, are phenyl.

The term “aralkyl” is used herein to denote a lower alkyl group, i.e. a cyclic, branched or straight chain alkyl group having one to six carbon atoms, in which there is an aryl substituent. A benzyl group is an especially preferred aralkyl substituent.

The term “substituted aryl” is used herein to denote aryl groups optionally substituted with one or more functional groups. Preferably, the “substituted aryl” is a phenyl group substituted with one or more (most preferably one to three) functional groups.

The term “substituted aralkyl” is used herein to denote aralkyl groups optionally substituted with one or more functional groups. Preferably, the “substituted aralkyl” is a benzyl group substituted with one or more (most preferably one to three) functional groups.

The term “heteroaryl” is used herein to denote an aryl substituent that comprises at least one hetero atom, such as N, O or S, in said at least one aromatic ring. A preferred heteroaryl substituent is pyridyl.

The term “heteroaralkyl” is used herein to denote an aralkyl substituent in which said at least one aromatic ring includes at least one hetero atom selected from, for example N, O or S. A preferred “heteroaralkyl” substituent is pyridylmethyl.

The terms “substituted heteroaryl” and “substituted heteroaralkyl” are used herein to denote heteroaryl and heteroaralkyl groups respectively that are substituted with one or more functional groups.

The term “halo” is used herein to denote a fluorine, chlorine, bromine or iodine atom.

Compounds of particular interest are those in which the substituent D is NHZ₂, i.e where Z₁ is H, and where Z₂ is an aryl aromatic ring which is either unsubstituted or which has at least one substituent X′ selected from a C₁-C₆ linear or branched alkyl chain optionally substituted with one or more substituents selected from amino, halo, cyano, OH or C₁-C₄ alkoxy; an unsubstituted or substituted aryl or aralkyl; halo; OH; SH; C₁-C₆ acyl; OR¹; SR¹; NR²R³; (CH₂)_{q}R²R³; NO₂; N₃; CN; O(CH₂)_{q}O in which the (CH₂)_{q} moiety is optionally substituted by one or more functional groups; COR⁴; CONR⁵R⁶; (CH₂)_{q}CONR⁵R⁶; COOR⁴; SOR⁴; SO₂R⁴ and SO₂NR⁵R⁶;

where R¹, R² and R³ each represent independently a C₁-C₆ linear or branched alkyl which is optionally substituted with one or more substituents selected from amino, OH, halo or a C₁-C₄ alkoxy group; and R⁴, R⁵ and R⁶ represent independently H; a C₁-C₆ linear or branched alkyl optionally substituted with one or more substituents selected from halo, OH, C₁₋₄ alkoxy and NR⁷R⁸; C₃-C₈ cycloalkyl; or an unsubstituted or substituted aralkyl, aryl or a group that comprises an unsubstituted or substituted 5- to 7-membered heterocyclic ring; and where q is in the range 1 to 6 and R⁷ and R⁸ represent independently H, a C₁-C₆ linear or branched alkyl optionally substituted with OH, amino or substituted amino.

Preferably, Z₂ is a substituted aryl comprising one to three substituents X′. In addition, in compounds of the invention where any of X′, R¹, R², R³, R⁴, R⁵ or R⁶ represent an optionally substituted C₁-C₆ linear or branched alkyl, the alkyl group is preferably provided with either one, two or three substituents. In compounds where any of X, R¹, R², R³, R⁴, R⁵ or R⁶ are halo substituted alkyl groups, the alkyl group may comprise either one, two or three halo substituents although alkyl groups comprising three halo substituents, such as CF₃ or CH₂CF₃, are preferred.

In compounds where R⁴, R⁵ and R⁶ independently represent a group comprising a heterocyclic ring, the heterocyclic ring preferably comprises at least one heteroatom selected from the group consisting of N, O and S. Preferably, the ring is a 5- to 7-membered heterocyclic ring. In addition, the heterocyclic ring may be either an aromatic ring or non-aromatic ring. Groups comprising an aromatic heterocyclic ring are preferably heteroaryl or heteroaralkyl groups whereas groups comprising a non-aromatic heterocyclic ring could be a heteroalicyclic group (e.g. tetrahydrofuran-2-yl) or a heteroalicyclicalkyl group (e.g. tetrahydrofuran-2-ylmethyl). The ring may also be optionally substituted with one or more substituents selected from a C₁-C₄ alkyl, OH, halo, amino or C₁-C₄ alkoxy.

In certain compounds of the present invention, R⁵ and R⁶ are linked to form a 5- to 7-membered heterocyclic ring. In addition to the nitrogen atom to which both the R⁵ and R⁶ groups are attached, the heterocyclic ring may also comprise one or more additional heteroatoms selected from N, O or S. Examples of heterocyclic ring structures that are formed when R⁵ and R⁶ are linked include morpholino and piperidino ring structures.

In embodiments of the invention where R⁴, R⁵ or R⁶ represent a C₁-C₆ linear or branched alkyl substituted with a NR⁷R⁸ group, both of the substituents R⁷ and R⁸ may be fused to form a 5- to 7-membered heterocyclic structure. In addition to the nitrogen atom to which both R⁷ and R⁸ are attached, the ring heterocyclic ring may also comprise one or more additional heteroatoms selected from the group of N, O or S. As above, examples of heterocyclic ring structures that are formed when R⁷ and R⁸ are linked include morpholino and piperidino ring structures.

In certain embodiments, Z₂ might be provided with two substituents X′ provided on adjacent carbon atoms and which are fused to form a cyclic substituent. In such compounds, Z₂ may, for example, be a structure represented by either of the structural formulae II or III below:

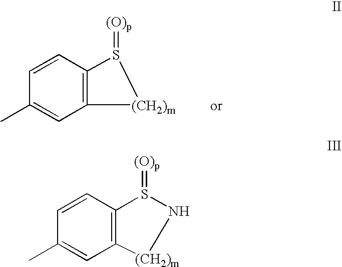

where p=1 or 2 and m is in the range of 2 to 5.

In many preferred embodiments Z₂ will be a substituted phenyl group substituted for example at position 4′ by a halogen atom or, more preferably, by a hydroxyl, hydroxyalkyl, an acetyl, a carbamoyl or by a sulfonamide group. Substituents X′ which are COR⁴, CONR⁵R⁶, COOR⁴, SOR⁴, SO₂R⁴ or SO₂NR⁵R⁶ as specified above are regarded as being particularly important for providing some of the most active CDK inhibiting compounds. Usually it is to be preferred that these substituents should be in the para or 4′-position. When the substituent in such phenyl group (or other aryl group) is a sulfonamide group this may itself be substituted in its amino group.

At least in most of the aforesaid compounds which are stated to be of particular interest can be represented by the following structural formula IV below:

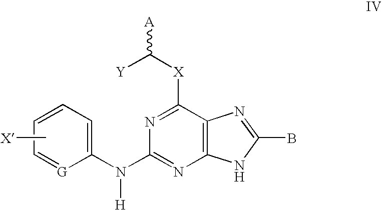

wherein G is CH or N, X′ is H or as defined above and the other substituents (A, B, X, Y) are as already defined.

Many of the compounds will be 2-anilinopurines and some representative examples of the structures are depicted below:

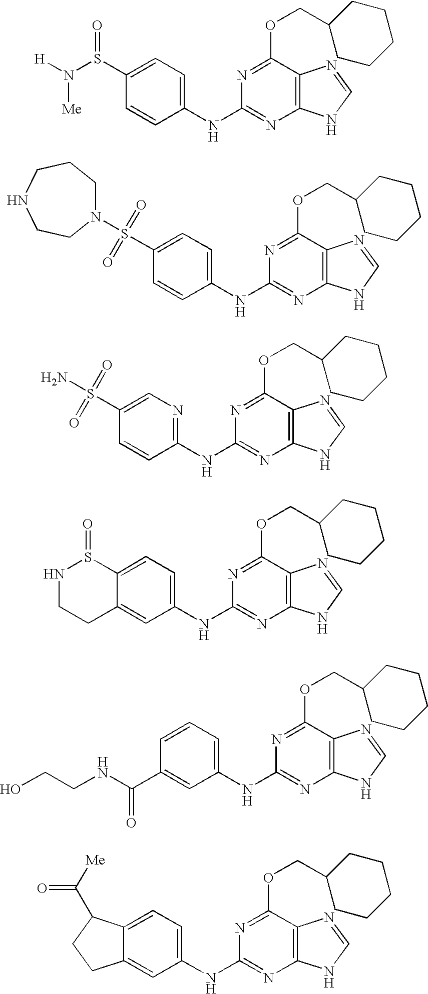

In some cases the CDK inhibitory activity of the purine compounds of this invention has been found to be selective towards different CDK's which is notably different from that of olomoucine.

So long as it is able to fit or seat in the ATP ribose binding pocket of a CDK protein and permit binding to the latter it is believed that there is a wide range of substituents likely to be suitable for Y and its precise composition is unlikely to be critical. In some cases it may be helpful for Y to comprise a ring structure that includes polar hydroxyl substituents or the like.

In most embodiments Y will be a cycloalkane or cycloalkene ring, preferably a 5- or 6-membered ring having up to two double bonds. Cyclohexyl and cyclophenyl is of particular interest. One or two carbon atoms in the ring may be replaced, however, by hetero atoms or groups, particularly O, S, NR′ (where R′ is H or C₁₋₄ alkyl) or, in a cycloalkene ring, —N═. Where the ring is substituted the substituent or each substituent (at any position) will preferably be selected from C₁₋₄ alkyl, OH, C₁₋₄ alkoxy, halogen, CF₃, CN, N₃ and NR_{y1}R_{y2} where R_{y1}, and R_{y2} are each independently H or C₁₋₄ alkyl. Moreover, in the case where there are two substituents on adjacent atoms of the ring,

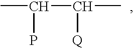

these substituents P and Q may be linked to form an additional fused ring structure, e.g. a 4-, 5- or 6-membered carbocyclic or heterocyclic ring. This additional ring structure may include, for example, up to two hetero atoms or groups such as O, S or NH, and it may also be substituted by one or more substituents, e.g. a C₁₋₄ alkyl group or groups or a phenyl or substituted phenyl group. In some embodiments, Y may also be adamantyl.

Examples of ring structures represented by Y include

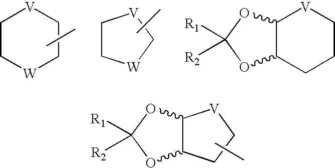

where V and W are each selected independently from the group consisting of O, S, NR′ (R′ is H or C₁₋₄ alkyl), CH₂ and/or ═CH—; and

R₁ and R₂ are each H or C₁₋₄ alkyl.

As indicated above, these ring structures represented by Y can optionally bear substituents which may be the same or different and which may inter alia be selected from C₁₋₄ alkyl, alkoxy, —OH, NR_{y1}R_{y2} (where R_{y1} and R_{y2} are each independently H or C₁₋₄ alkyl), CF₃, halogen, N₃, CN, optionally substituted aryl (e.g. phenyl), and optionally substituted aralkyl (e.g. benzyl). Also, as already indicated, it may be useful, e.g. to improve solubility, in some cases for the ring structure to include a plurality of polar substituents such as hydroxyl for example.

It will be understood that where reference is made in this specification to compounds of formula I such reference should be construed as extending also to their pharmaceutically acceptable salts and to other pharmaceutically acceptable bioprecursors (prodrug forms) where relevant. The term “prodrug” is used in the present specification to denote modified forms or derivatives of a pharmacologically active compound which biodegrade or are modified in vivo so as to become converted into said active compound after administration, especially oral or intravenous administration, in the course of therapeutic treatment of a. mammal. Such prodrugs are commonly chosen because of an enhanced solubility in aqueous media which helps to overcome formulation problems, and also in some cases to give a relatively slow or controlled release of the active agent.

It should also be understood that where any of the compounds referred to can exist in more than one enantiomeric and/or diastereoisomeric form, all such forms, mixtures thereof, and their preparation and uses are within the scope of the invention. It should be noted, however, that stereochemical considerations are likely to be important and there may be considerable selectivity such that different enantiomers or diastereoisomers have significantly different inhibitory activity.

Preferably, in compounds in accordance with structural formula I used in carrying out the invention, X will be oxygen. Also, each alkyl group present, either as such or in a moiety in an alkoxy or other group, contains 1-6 carbon atoms unless otherwise specified.

Although it will usually be preferred that Y should comprise a saturated or partially saturated carbocyclic or heterocyclic ring structure, it should be recognised that in some cases Y may comprise an aromatic ring system (e.g. optionally substituted aryl or aralkyl), and still provide compounds of interest as potentially selective CDK inhibitors that may be useful in the context of the present invention.

Examples of compounds which are at present of especial interest or preferred for use in carrying out the invention and which include the most potent CDK inhibitors that have been identified, at least when assayed in vitro against CDK1 and/or CDK2, comprise the following:

2-Anilino-6-cyclohexylmethoxypurine

2-(3′-Bromoanilino)-6-cyclohexylmethoxypurine

6-Cyclohexylmethoxy-2-(3′-chloroanilino)purine

6-Cyclohexylmethoxy-2-(4′-fluoroanilino)purine

6-Cyclohexylmethoxy-2-(3′-fluoroanilino)purine

6-Cyclohexylmethoxy-2-(3′-ethylanilino)purine

6-Cyclohexylmethoxy-2-(4′-methoxyanilino)purine

6-Cyclohexylmethoxy-2-(3′-methoxyanilino)purine

6-Cyclohexylmethoxy-2-(3′-methylmercaptoanilino)purine

6-Cyclohexylmethoxy-2-(4′-methylmercaptoanilino)purine

Methyl 4-N-(6-cyclohexyl-methoxypurin-2-yl)aminobenzoate

6-Cyclohexylmethoxy-2-(3′-chloro-4′-fluoroanilino)purine

6-Cyclohexylmethyloxy-2-(4′-hydroxyanilino)purine

4-(6-Cyclohexyl-methoxylpurin-2-yl)amino-acetophenone

6-Cyclohexylmethoxy-2-(4′-sulfamoylanilino)purine

N-Benzyl-4-(cyclohexylmethoxypurine-2-ylamino)-benzenesulfonamide

4-[(6-Cyclohexylmethoxy-purine-2-ylamino)methyl]-benzenesulfonamide

Methyl-4-[(6′-cyclohexylmethoxylpurin-2-yl)amino]phenylsulfoxide

4-(6-Cyclohexylmethoxy-purin-2-yl)amino)-N-methyl-benzenesulfonamide

4-(6-Cyclohexylmethoxy-purin-2-ylamino)-N,N-dimethylbenzenesulfonamide

4-(6-Cyclohexylmethoxypurin-2-ylamino)-N-ethyl-benzenesulfonamide

4-(6-Cyclohexylmethoxypurin-2-ylamino)-N-isobutyl-benzene-sulfonamide

4-(6-Cyclohexylmethoxy-9H-purin-2-ylamino)-N-cyclopropyl-benzenesulfonamide

N-(3-Chlorophenyl)-4-(6-Cyclohexylmethoxy-9H-purin-2-ylamino)-benzenesulfonamide

4-(6-Cyclohexylmethoxy-9H-purin-2-ylamino)-N-propyl-benzenesulfonamide

4-(6-Cyclohexylmethoxy-9H-purin-2-ylamino)-N-(tetrahydrofuran-2-ylmethyl)-benzenesulfonamide

4-(6-Cyclohexylmethoxy-9H-purin-2-ylamino)-N-(2-methoxy-ethyl)-benzenesulfonamide

4-(6-Cyclohexylmethoxy-9H-purin-2-ylamino)-N,N-dimethyl-benzamide

4-(6-Cyclohexylmethoxy-9H-purin-2-ylamino)-N-methylbenzamide

4-(6-Cyclohexylmethoxy-9H-purin-2-ylamino)-N,N-diethyl-benzamide

4-(6-Cyclohexylmethoxy-9H-purin-2-ylamino)-benzamide

4-(6-Cyclohexylmethoxy-9H-purin-2-ylamino)-N-propyl-benzamide

4-(6-Cyclohexylmethoxy-9H-purin-2-ylamino)-N-ethyl-benzamide

4-(6-Cyclohexyloxymethoxy-9H-purin-2-ylamino)-N-(2-hydroxy-ethyl)-benzenesulfonamide

4-(6-Cyclohexyloxymethoxy-9H-purin-2-ylamino)-N-thiazol-2-yl-benzenesulfonamide

4-(6-Cyclohexyloxymethoxy-9H-purin-2-ylamino)-N-(2-hydroxy-propyl)-benzenesulfonamide

4-(6-Cyclohexyloxymethoxy-9H-purin-2-ylamino)-benzoic acid

[3-(6-Cyclohexyloxymethoxy-9H-purin-2-ylamino)-phenyl]methanol

4-(6-Cyclohexylmethoxy-9H-purin-2-ylamino)-N-phenylbenzene-sulfonamide

4-(6-Cyclohexylmethoxy-9H-purin-2-ylamino)-N-(2,3-dihydroxypropyl)-benzenesulfonamide

4-(6-Cyclohexylmethoxy-9H-purin-2-ylamino)-(N-pyridin-2-yl)-benzenesulfonamide

4-(6-Cyclohexylmethoxy-9H-purin-2-yl)-(4-methane-sulfonylphenyl)amine

4-(6-Cyclohexylmethoxy-9H-purin-2-ylamino)-N-(3-diethylaminopropyl)-benzenesulfonamide

4-[6-(-2-Methyl-cyclohexylmethoxy)-9H-purin-2-ylamino]benzenesulfonamide

4-(6-Cyclohexylmethoxy-9H-purin-2-ylamino)-N-(2,6-dimethylpyrimidin-4-yl)-benzenesulfonamide

4-[6-Cyclohexylmethoxy-9H-purin-2-ylamino]-benzene(3′-methoxy)sulfonamide

4-[6-(Cyclohex-3-enylmethoxy)-9H-purin-2-ylamino]-benzenesulfonamide

4-(6-Cyclohexylmethoxy-9H-purin-2-ylamino)-N-(2,2,2-trifluoroethyl)-benzenesulfonamide

2-[4-(6-Cyclohexylmethoxy-9H-purin-2-ylamino)]-phenylacetamide

4-[6-Cyclopentylmethoxy-9H-purin-2-ylamino)-benzenesulfonamide

4-(6-Cyclohexylmethoxy-9H-purin-2-ylamino)-N-(2-hydroxypropyl)benzenesulfonamide

Biological Activity

Assays are available for testing the inhibitory activity of the compounds of interest against a range of CDK/cyclin complexes, including CDK1/cyclin A, CDK1/cyclin B, CDK1/cyclin F, CDK2/cyclin A, CDK2/cyclin E, CDK4/cyclin D, CDK5/35 and CDK6/cyclin D3. It is of particular interest to note the selectivity of some of the compounds against different CDK's.

Test results showing CDK inhibitory activity values measured for some of the purine compounds in accordance with the present invention that have been prepared are shown in Table 1 at the end of the present description. Where the compounds exist in different enantiomorphic forms, the assays have generally been carried out on racemic mixtures. Apart from reference compounds, the compounds listed are accompanied by an NU reference or identification code number. Table 1 includes the compounds which at present are the most preferred of those that have been prepared, although as yet not all have been fully tested.

In general, the studies carried out fully support the belief that CDK inhibitory characteristics of compounds tested reflect an ability of these compounds to act as effective antitumour drugs.

The inhibition assays have been carried out using methods based on those described in the paper hereinbefore referred to of J. Vesely et al and in the paper of L. Azzi et al (1992) Eur. J. Biochem. 203, 353-360. By way of example, however, a typical protocol is summarised below.

CDK Assay Example

Reagents:

Buffer C (containing 6 mM β-glycerophosphate, 30 mM nitrophenyl phosphate, 25 mM MOPS pH 7.0, 5 mM EGTA, 15 mM MgCl₂, 1 mM MgCl₂ and 0.1 mM sodium orthovanadate) is made up as follows:

| | FW | g/100 ml | Final conc |
|---|---|---|---|
| β-glycerophosphate (RT) | 216 | 1.3 | 60 mM |
| MOPS (RT) | 209.3 | 0.52 | 25 mM |
| EGTA (RT) | 380.4 | 0.19 | 5 mM |
| MgCl₂ (RT) | 203.4 | 0.305 | 15 mM |

(a) First dissolve above ingredients in about 80 ml distilled water and pH to 7.0

(b) Then add 1 ml 10 mM sodium orthovanadate

(1.84 mg/ml-FW=183.9 RT)

final conc=0.1 mM, and cool to 4° C.

(c) Then Add

| | | | |
|---|---|---|---|
| 4-nitrophenyl phosphate (−20° C.) | 279.2 | 1.112 | 30 mM |
| DTT (4° C.) | 154.2 | .0154 | 1 mM |

(Alternatively, make up 100 mM DTT (15.4 mg/ml) and store in 1.2 ml aliquots in freezer, thaw and add 1 ml to buffer, above)

(d) Make up to 100 ml and store in 5 ml aliquots in freezer.

Affinity purified p34 cdc2(CDK1)/cyclinB from M-phase starfish (Marthasterias glacialis) in 20% glycerol is stored at −80° C. in chest freezer

100 mM Olomoucine (Cat # LC-0-3590-M025 Alexis Co. Bingham Nottingham). FW=298.35 29.835 mg/ml=100 mM, is stored in freezer 25 ml aliquots.

1% phosphoric acid (58.8 ml 85% phosphoric acid+4.942 litres water)

Make up the following on day of assay:

Histone H1 (type III-S (Sigma) 4° C.) 5 mg/ml in buffer C.

[³²P]ATP 75 mM: Make up using (multiples of) the following proportions:

2 ml [³²P]ATP (3000Ci/mMol PB168 Amersham, stored in radioactive freezer)+7.5 ml 1 mM cold ATP (−20° C.) (0.551 mg/ml-200 ml aliquots stored in freezer)+90.5 ml buffer C

Conc.=12.5 mM in final assay

Assay Procedure

DMSO cannot exceed 1% in the assay mixture. Inhibitors are added at 1/10 final assay volume and 10× final strength. DMSO stocks must therefore be diluted to 10× final desired concentration in ≦10% DMSO, ≧90% buffer C. Suggested concentration ranges=0, 1, 10, 100 mM so DMSO stocks of 0, 100, 1,000 and 10,000 mM are diluted 1/10 in buffer C before adding to assay.

Preparation

Label set of 0.2 ml microtubes for assay (e.g. A₀, A₁, A₁₀, A₁₀₀) in suitable rack and another set of Eppendorf™ vessels for drug dilution

Label phosphocellulose filters in pencil (e.g. A₀, A₁, A₁₀, A₁₀₀) and fold longitudinally to make a “pitched roof”

Set up water bath at 30° C. containing second rack for microtubes

Set up beaker containing wire mesh insert and magnetic flea below mesh insert, together with 400 ml 1% phosphoric acid, on magnetic stirrer

Reaction Mix:

All reagents (except DMSO stocks) should be kept on ice until assay initiated.

Place rack of assay tubes on ice

In each tube put:

16 ml buffer C

1 ml cdc2/cyclinB kinase

5 ml histone H1

3 ml inhibitor

Start reaction in each tube at 30 second intervals b)y adding

5 ml [³²P]ATP vortexing and placing in rack in waterbath at 30° C.

Terminate reaction after 10 min at 30 second intervals in tubes in same order by removing 25 ml reaction mix and spotting onto appropriately labelled filter, allowing to dry for 20-30 seconds and transferring to stirring 1% phosphoric acid.

Blank incubation is performed as above but without histone (add 5 ml buffer C instead) Washing blank is 5 ml ATP added directly to filter.

Wash filters 5-6 times 5 min each

Dry the filters on paper towel

Count in mini scintillation vials with 5 ml scintillant.

3×standards of 5 ml ATP counted also (375 pmoles ATP)

NB. The assay can be simplified by making up stock reaction mix as follows:

(1 part cdc2/cyclinB, 16 parts buffer C, 5 parts histone H1)×Number of assay tubes+1 and add 22 ml to each assay tube containing 3 ml buffer C±inhibitor.

It is still necessary, however, to make up assay blank (i.e. without histone) separately.

Therapeutic Use

The present invention also relates to the therapeutic utility of the purine compounds previously defined. Thus, in a further aspect, the present invention provides a purine compound as herein before defined for use in therapy. More specifically, the present invention also provides a purine compound as hereinbefore defined for use as an active pharmaceutical substance for the treatment of tumours or other cell proliferation disorders.

In yet another further aspect of the invention, there is provided the use of purine compounds as hereinbefore defined in the manufacture of a medicament for the treatment of tumours or other cell proliferation disorders.

As already indicated, compounds of this invention can inhibit tumor cell proliferation and may have significant selective antitumor activity. Antitumor activity may be evidenced by reduction of tumor cell number in mammals bearing cancer tumors, e.g. breast cancer tumors, and a consequent increase in survival time as compared to a control provided by animals which are untreated. Antitumor activity is further evidenced by measurable reduction in the size of solid tumors following treatment with the compounds of this invention compared to the tumors of untreated control animals.

Accordingly, the compounds of the present invention are of particular interest for the treatment of a range of selected cancer tumors, and the invention further provides a method for the treatment of a patient suffering from certain kinds of cancer. For this purpose, a therapeutically effective non-toxic amount of a compound of formula (I) as hereinbefore defined, may be suitably administered, orally, parenterally (including subcutaneously, intramuscularly and intravenously), or topically. The administration will generally be carried out repetitively at intervals, for example once or several times a day.

The amount of the compound of formula (I) which is required in order to be effective as an antitumor agent for treating mammals will of course vary and is ultimately at the discretion of the medical or veterinary practitioner treating the mammal in each particular case. The factors to be considered by such a practitioner, e.g. a physician, include the route of administration and pharmaceutical formulation; the mammal's body weight, surface are age and general condition; and the chemical form of the compound to be administered. However, a suitable effective antitumor dose may be in the range of about 1.0 to about 75 mg/kg bodyweight, preferably in the range of about 5 to 40 mg/kg with most suitable doses being for example in the range of 10 to 30 mg/kg. In daily treatment for example, the total daily dose may be given as a single dose, multiple doses, e.g. two to six times per day, or by intravenous infusion for any selected duration. For example, in the case of a 75 kg mammal, the dose range could be about 75 to 500 mg per day, and it is expected that a typical dose would commonly be about 100 mg per day. If discrete multiple doses are indicated, treatment might typically be 50 mg of the compound of formula (I), given 4 times per day in the form of a tablet, capsule, liquid (e.g. syrup) or injection.

While it may be possible for the compounds of formula (I) to be administered alone as the raw chemical, it is preferable to present the compounds in a pharmaceutical composition. Thus, the invention also provides pharmaceutical compositions containing an effective CDK-inhibiting non-toxic amount of a purine compound as defined above which forms the active therapeutic ingredient. Such pharmaceutical compositions for medical use will be formulated in accordance with any of the methods well known in the art of pharmacy for administration in any convenient manner. The CDK-inhibiting compounds will usually be admixed with at least one other ingredient providing a compatible pharmaceutically acceptable additive, carrier, diluent or excipient, and may be presented in unit dosage form.

The carrier(s) must be pharmaceutically acceptable in the sense of being compatible with the other ingredients of the formulation and not deleterious to the recipient thereof.

The possible formulations include those suitable for oral, rectal, topical and parenteral (including subcutaneous, intramuscular and intravenous) administration or for administration to the lung or another absorptive site such as the nasal passages.

All methods of formulation in making up such pharmaceutical compositions will generally include the step of bringing the compound of formula (I) into association with a carrier which constitutes one or more accessory ingredients. Usually, the formulations are prepared by uniformly and intimately bringing the compound of formula (I) into association with a liquid carrier or with a finely divided solid carrier or with both and then, if necessary, shaping the product into desired formulations.

Formulations of the present invention suitable for oral administration may be presented as discrete units such as capsules, cachets, tablets or lozenges, each containing a predetermined amount of the compound of formula (I); as a powder or granules; or a suspension in an aqueous liquid or non-aqueous liquid such as a syrup, an elixir, an emulsion or a draught. The compound of formula (I) may also be presented as a bolus, electuary or paste.

A tablet may be made by compression or moulding, optionally with one or more accessory ingredients. Compressed tablets may be prepared by compressing, in a suitable machine, the compound of formula (I) in a free-flowing form such as a powder or granules, optionally mixed with a binder, lubricant, inert diluent, surface active or dispersing agent. Moulded tablets may be made by moulding, in a suitable machine, a mixture of the powdered compound of formula (I) with any suitable carrier.

A syrup may be made by adding the compound of formula (I) to a concentrated, aqueous solution of a sugar, for example sucrose, to which may be added any desired accessory ingredient. Such accessory ingredient(s) may include flavourings, an agent to retard crystallisation of the sugar or an agent to increase the solubility of any other ingredient, such as a polyhydric alcohol, for example glycerol or sorbitol.

Formulations for rectal administration may be presented as a suppository with a usual carrier such as cocoa butter.

Formulations suitable for parental administration conveniently comprise a sterile aqueous preparation of the compound of formula (I) which is preferably isotonic with the blood of the recipient.

In addition to the aforementioned ingredients, formulations of this invention, for example ointments, creams and the like, may include one or more accessory ingredients, for example a diluent, buffer, flavouring agent, binder, surface active agent, thickener, lubricant and/or a preservative (including an antioxidant) or other pharmaceutically inert excipient.

The compounds of this invention may also be made up for administration in liposomal formulations which can be prepared by methods well-known in the art.

Thus, it will be appreciated that the invention also includes the use of the CDK-inhibiting purine compounds defined above for the manufacture of medicaments or pharmaceutical compositions for treating tumours or other cell proliferation disorders wherein the said purine compound itself provides an effective independent antitumour or cell proliferation inhibiting agent.

The invention also includes the treatment of abnormal cellular proliferation disorders using such medicaments or pharmaceutical compositions.

### DESCRIPTION OF ILLUSTRATIVE EXAMPLES

The following examples and description of stages in synthetic routes of preparation of various exemplary compounds of interest serve further to illustrate the present invention, but should not be construed in any way as a limitation thereof. Again, in many instances the compounds described are accompanied by an NU reference or identification code number.

For the purpose of illustration only, 6-cyclohexylmethoxy purine derivatives have been chosen to illustrate the invention.

In the next section the preparation of a range of active purine derivatives in accordance with the invention is described. The first eight compounds of which the preparation is described in EXAMPLES 1 to 8, however, are primarily intermediate compounds used in the subsequently described preparation of a number of the other active compounds. More particularly, EXAMPLES 1 to 5 describe the preparation of various aniline derivatives that may be used in the preparation of certain embodiments of the invention.

All the active purine compounds herein described in the specific examples are in fact prepared using the compound O⁶-cyclohexylmethyl-2-fluoropurine (NU6061), usually employing one of four general procedural methods designated “A”, “B”, “C” and “D”. These general procedures or methods for preparation of these purine derivatives are described below.

Preparation of the Purine Derivatives. General Procedure A.

A solution of O⁶-cyclohexylmethyl-2-fluoropurine in either EtOH or n-BuOH was stirred at room temperature under nitrogen, and the appropriate aniline (7 equiv.) and trifluoroacetic acid (TFA) (0.4 equiv.) were added dropwise. The solution was heated, with stirring, at the specified temperature until TLC analysis [silica, 1:9 (v/v) MeOH/DCM] confirmed reaction completion. The reaction mixture was cooled, and the product was collected by filtration and recrystallised from the appropriate solvent.

Preparation of the Purine Derivatives. General Procedure B.

To a stirred solution of O⁶-cyclohexylmethyl-2-fluoropurine in either n-BuOH or a mixture of n-BuOH and glycerol, was added the appropriate aniline derivative (4 equiv.) and trifluoroacetic acid (10 equiv.). The solution was heated under nitrogen to the specified temperature until TLC analysis [silica, 1:9 (v/v) MeOH/DCM] confirmed reaction completion. The mixture was cooled and the solvents were removed in vacuo. Water was added and the solution was neutralised with NaHCO₃, extracted with EtOAc, and dried (MgSO₄). The combined organic layers were evaporated in vacuo to give the required compound, which was recrystallised from the appropriate solvent.

Preparation of Purine Derivatives. General Procedure C.

A mixture of the appropriate aniline (2 equiv) and TFA (5 equiv.) in glycerol was added dropwise to a solution of O⁶-cyclohexylmethyl-2-fluoropurine in n-BuOH. The solution was heated with stirring to the specified temperature until TLC analysis confirmed reaction completion [silica, 1:9 (v/v) MeOH/DCM]. The mixture was cooled and the volatiles were removed in vacuo. The residue was dissolved in water and the solution was neutralised with NaHCO₃, extracted with EtOAc and dried (MgSO₄). The combined organic layers were evaporated in vacuo to give the desired compound, which was purified by chromatography on silica.

Preparation of the Purine Derivatives. General Procedure D.

To a solution of O⁶-cyclohexylmethyl-2-fluoropurine in a mixture of glycerol, and either n-BuOH or EtOH, was added the appropriate aniline derivative (7 equiv.) and TFA (0.4 equiv.) dropwise at the same time. The solution was heated to the specified temperature until TLC analysis [silica, 1:9 (v/v) MeOH/DCM] confirmed reaction completion. After cooling, the solvent was removed in vacuo, water was added and the solution was neutralised with NaHCO₃. The solution was extracted with EtOAc, dried (MgSO₄) and the combined organic layers were evaporated in vacuo to give the desired compound, which was purified by chromatography on silica.

There now follows descriptions of the preparation of specific examples.

### EXAMPLES

### EXAMPLE 1

Preparation of 1-(4-Aminophenyl)ethanone

Prepared from 1-(4-nitro-phenyl)ethanone using SnCl₂ (5 equiv.) in EtOH to reflux. After 4 h, the solution was cooled (ice bath), neutralised using NaOH, and extracted with EtOAc. The combined organic layers were dried (MgSO₄) and concentrated in vacuo to lead to the crude compound.

### EXAMPLE 2

Preparation of 4-aminobenzenesulfonamides

A suspension of N-4-acetylbenzenesulfonyl chloride (1 equiv.) in EtOH was added to the appropriate amine (2 equivalents) and the solution was heated to 60° C. After 30 min, the mixture was cooled (ice bath) and water was added to precipitate the crude product, which was collected and washed with water. The compound obtained was added to a solution of HCl in EtOH and heated to reflux for 5 h. The solution was then successively washed with water and a NaHCO₃ solution (2.0 M) and the crude compound extracted into dichloromethane (DCM). The organic layer was dried (MgSO₄) and concentrated in vacuo to afford the required 4-aminobenzenesulfonamides.

### EXAMPLE 3

Preparation of 4-aminobenzamides

A mixture of the appropriate amine (1 equiv.) and triethylamine (1.2 equiv.) in CHCl₃ was added dropwise, with stirring, to an ice cooled solution of 4-nitrobenzoyl chloride (1.1 equiv.). The mixture was stirred for 3 h at room temperature and was successively washed with water, NaHCO₃ solution (2 M), and water. The organic layer was dried (MgSO₄) and concentrated in vacuo to give the crude compound, which was redissolved in a mixture of aqueous HCl and AcOH. SnCl₂ (4 equiv.) was added and the mixture was heated under reflux for 3 h. The solution was cooled (ice bath), neutralised with NaOH solution, and extracted with EtOAc. The combined organic layers were dried (MgSO₄), and evaporated in vacuo to give the required 4-aminobenzamides derivative.

### EXAMPLE 4

4-(Methanesulfinyl)phenylamine

Hydrogen peroxide (27% v/v in water) containing urea-hydrogen peroxide (in excess) was added to a solution of 4-methylsulfanylphenylamine in acetone. The mixture was stirred at 60° C. for 10 h, cooled to room temperature, and extracted with DCM. The combined organic layers were dried (MgSO₄) and the solvent was evaporated in vacuo to furnish the required sulfoxide.

### EXAMPLE 5

4-Methanesulfonyl)phenylamine

To a solution of 4-methylsulfanylphenylamine in aqueous acetic acid (60% w/v) was added sulphuric acid (3 M). Hydrogen peroxide solution (27% aqueous) was added dropwise to the mixture until a precipitate was observed to develop. The mixture was stirred for 15 min, cooled to room temperature and diluted with water. The solution was extracted with DCM, dried (MgSO₄) and the combined organic layers were concentrated in vacuo to yield the required sulfone.

### EXAMPLE 6

2-Amino-6-(1,4-diazabicyclo[2.2.2]oct-1-yl)purinium chloride (‘DABCO-purine’)

To a solution of 2-amino-6-chloropurine (1 equiv.) in DMSO (24 mL/g starting material) was added 1,4-diazabicyclo[2.2.2]octane (5.5 equiv.) in portions with stirring. The reaction mixture was stirred at room temperature for 24 h, completion of the reaction being confirmed by TLC [silica, 1:9 (v/v) MeOH/DCM]. The bulk of the solvent was removed under reduced pressure, and the last traces of DMSO were removed by triturating with Et₂O. The solid was collected by filtration, redissolved in H₂O and extracted into EtOAc. The water layer was evaporated to dryness to give a white solid (97%), m.p. >230°C.

### EXAMPLE 7

2-Amino-O⁶-cyclohexylmethylpurine (NU2058)

Cyclohexylmethanol (4 equiv.) was added dropwise to a stirred suspension of NaH (3 equiv.) in anhydrous DMSO at room temperature. After 1 h, DABCO-purine (1 equiv.) was added in portions over 5 min. The reaction mixture was stirred at room temperature for 4-5 days, reaction completion being confirmed by TLC [silica, 1:9 (v/v) MeOH/DCM]. The reaction mixture was neutralised using glacial acetic acid, water was added, and the solution was extracted with EtOAc. The combined organic layers were evaporated in vacuo, and the crude product was washed with DCM to remove any remaining Cyclohexylmethanol, affording the title compound as a white powder (93%), m.p. 191-193° C.

### EXAMPLE 8

O⁶-Cyclohexylmethyl-2-fluoropurine (NU6061)

To a stirred solution of 2-amino-O⁶-cyclohexylmethylpurine (NU2058) in 48% aqueous fluoroboric acid (25 equiv. HBF₄) at −12° C., was added a solution of NaNO₂ (0.3 M, 2 equiv.) in water dropwise over 80 min. The resulting solution was allowed to warm to room temperature. After 1 h, the solution was cooled to 0° C. and neutralised to pH 7 with conc. aq. NaOH. The solution was evaporated to dryness under reduced pressure, and the residual white solid was purified either by flash chromatography on silica employing DCM:MeOH (9:1) as eluent, or by Soxhlet extraction employing diethyl ether as solvent. The title compound was obtained as a white powder (44%), m.p. 232-233° C.

### EXAMPLE 9

2-Anilino-6-cyclohexylmethoxypurine (NU6094)

To a solution of O⁶-cyclohexylmethyl-2-fluoropurine (1 equiv.) in n-BuOH was added aniline (5 equiv.), and the mixture was heated at 120° C. for 16 h. The solvent was removed in vacuo, DCM was added, and the resulting compound was filtered to give a white solid. The product was purified by chromatography on silica using DCM, followed by DCM:MeOH (95:5) as the eluting solvent, to yield the title compound as a white solid (46%). Recrystallisation from MeOH gave the title compound, m.p. 196-199° C.

### EXAMPLE 10

2-(3′-Chloroanilino)-6-cyclohexylmethoxypurine (NU6086)

This was prepared according to general procedure A employing 3-chloroaniline and n-BuOH. The solution was heated to reflux for 24 h and the crude compound was precipitated by adding MeOH. Recrystallisation from MeOH afforded the title compound as a white solid (28%), m.p. 196-199° C.

### EXAMPLE 11

2-(3′-Bromoanilino)-6-cyclohexylmethoxypurine (NU6095)

Prepared according to general procedure A from 3-bromoaniline and n-BuOH. The solution was heated to reflux for 18 h and the crude compound was precipitated by adding MeOH. The product was purified by chromatography on silica, using DCM followed by DCM:MeOH (95:5) as the eluting solvents, to yield the title compound as a white solid (35%), m.p. 204-207° C.

### EXAMPLE 12

6-Cyclohexylmethoxy-2-(3′-methoxyanilino)purine (NU6096)

Prepared according to general procedure A using 3-methoxyaniline and n-BuOH. The solution was heated to reflux for 2 h and adding DCM precipitated the product. Recrystallisation from MeOH furnished the title compound as a white solid (40%), m.p. 186-189° C.

### EXAMPLE 13

6-Cyclohexylmethoxy-2-(3′-fluoroanilino)purine (NU6098)

Prepared according to general procedure A using 3-fluoroaniline and n-BuOH. The solution was heated to reflux for 1 h and adding MeOH precipitated the product as a white solid (50%), m.p. 224-227° C.

### EXAMPLE 14

6-Cyclohexylmethoxy-2-(3′-ethylanilino)purine (NU6099)

Prepared according to general procedure A using 3-ethylaniline and EtOH. The solution was heated to reflux for 1 h and the crude compound precipitated directly on cooling. Recrystallisation from MeOH afforded the title compound as a white solid (34%), m.p. 180-183° C.

### EXAMPLE 15

6-Cyclohexylmethoxy-2-(4′-methoxyanilino)purine (NU6100)

Prepared according to general procedure A using 4-methoxyaniline and EtOH. The solution was heated to reflux for 16 h and the product precipitated directly on cooling. Recrystallisation from MeOH gave the title compound as a white solid (41%), m.p. 189-192° C.

### EXAMPLE 16

6-Cyclohexylmethoxy-2-(3′-methylmercaptoanilino)purine (NU6101)

Prepared according to general procedure A using 3-methylmercaptoaniline and EtOH. The solution was heated to reflux for 16 h and the crude compound precipitated directly on cooling. Recrystallisation from MeOH gave the title compound as a white solid (44%), m.p. 190-192° C.

### EXAMPLE 17

6-Cyclohexylmethoxy-2-(4′-fluoroanilino)purine (NU6116)

Prepared according to general procedure A from 4-fluoroaniline and n-BuOH. The solution was heated to reflux for 16 h and the crude compound was precipitated on adding MeOH. Recrystallisation from MeOH gave the title compound as an off white solid (25%), m.p. 207-209° C.

### EXAMPLE 18

6-Cyclohexylmethoxy-2-(4′-methylmercaptoanilino)purine (NU6117)

Prepared according to general procedure A using 4-methylmercaptoaniline and n-BuOH. The solution was heated to reflux for 16 h and the crude compound was precipitated directly on cooling. Recrystallisation from EtOH and MeOH, respectively, gave the title compound as an off white solid (18%), m.p. 159-160° C.

### EXAMPLE 19

Methyl 4-N-(6′-Cyclohexylmethoxypurin-2′-yl)aminobenzoate (NU6118)

Prepared according to general procedure A using methyl-4-aminobenzoate and n-BuOH. The solution was heated to reflux for 16 h and the crude product was precipitated on addition of MeOH. Purified by chromatography on silica, using DCM:MeOH (9:1) as eluent, followed by recrystallisation from MeOH to give the title compound as an off white solid (8%), m.p. 244-246° C.

### EXAMPLE 20

6-Cyclohexylmethoxy-2-(3′-chloro-4′-fluoroanilino)purine (NU6119)

Prepared according to general procedure A using 3-chloro-4-fluoroaniline and n-BuOH. The solution was heated to reflux for 16 h and the crude compound was precipitated on addition of MeOH. Recrystallisation from MeOH gave the target compound as an off white solid (21%), m.p. 225-226° C.

### EXAMPLE 21

6-Cyclohexylmethoxy-2-(4′-hydroxyanilino)purine (NU6120)

Prepared according to general procedure A using 4-aminophenol and n-BuOH. The solution was heated to reflux for 16 h and the crude compound precipitated on addition of MeOH. Recrystallisation from MeOH afforded the target compound as an off white solid (51%). ¹H NMR (200 MHz, d⁶-DMSO) δₚₚₘ 1.31 (5H, m), 1.92 (6H, m), 4.40 (2H, d), 6.76 (2H, d), 7.62 (2H, d), 8.02 (1H, s), 9.04 (2H, s). MS (EI) 339.170807 (calculated as 339.169525) M⁺339 (53%), 243 (100%), m.p. >290° C. (decomp).

### EXAMPLE 22

6-Cyclohexylmethoxy-2-(4′-cyanomethylanilino)purine (NU6121)

Prepared according to general procedure A using 4-aminophenylacetonitrile and n-BuOH. The solution was heated to reflux for 16 h and the crude compound was precipitated on addition of MeOH. Recrystallisation from MeOH gave the target compound as an off white solid (17%). ¹H NMR (200 MHz, d⁶-DMSO) δₚₚₘ 1.31 (5H, m), 1.92 (6H, m), 4.04 (2H, s), 4 42 (2H, d), 7.34 (2H, d), 7.92 (2H, d), 8.11 (1H, s). MS (EI) 362.186272 (calculated as 362.185510) M⁺362 (55%), 266 (100%), m.p. 191-192° C.

### EXAMPLE 23

4-(6-Cyclohexylmethoxypurin-2-ylamino)benzoic acid (NU6147)

Prepared by the saponification of NU6118 as follows: a solution of KOH in EtOH (2 equiv.) was added dropwise to an ice cold solution of NU66118 in THF, and the mixture was stirred for 24 h. Water was added, and the solution was neutralised with aqueous HCl solution. The mixture was extracted with DCM and the combined organic layers were dried over MgSO₄ and evaporated in vacuo to give the title compound (41%). ¹H NMR (200 MHz, d⁶-DMSO) δppm 1.31 (5H, m), 1.93 (6H, m), 4 45 (2H, d), 8.02 (4H, q), 8.19 (1H, s), 9.92 (s, 1H), 13.05 (1H, s). MS (ESI+) m/z 368 (M+1).

### EXAMPLE 24

6-Cyclohexylmethoxy-2-(4′-sulfamoylanilino)purine (NU6102)

Prepared according to general procedure B using a mixture of n-BuOH/glycerol and sulfanilamide. The solution was heated to reflux for 4.5 days. The crude compound was washed with a mixture of Et₂O and petrol, and recrystallised from hot water to lead to give the title compound as a white solid (60%), m.p. 152-154° C.

### EXAMPLE 25

4-(6′Cyclohexylmethylpurin-2-yl)aminoacetophenone (NU6125)

Prepared according to general procedure B using n-BuOH and 1-(4-aminophenyl)ethanone (from Example 1). The solution was heated to reflux for 0.5 days. The crude product was washed with hot water to afford the title compound as a yellow powder (61%), m.p. 236-238° C.

### EXAMPLE 26

Methyl-4-[(6′-cyclohexylmethylpurin-2-yl)amino]phenylsulfoxide (NU6126)

Prepared according to general procedure B using n-BuOH and 4-(methanesulfinyl)phenylamine (from Example 4). The solution was heated to reflux for 2 days. The crude product was purified by chromatography on silica employing EtOAc-petrol (95:5), followed by DCM:MeOH (9:1), as eluents, giving the title compound as a brown solid (46%), m.p. 150-152° C.

### EXAMPLE 27

4-(6-Cyclohexylmethoxy-2-ylamino)-N-methylbenzenesulfonamide (NU 6127)

Prepared according to general procedure B using n-BuOH and 4-amino-N-methylbenzenesulfonamide (from Example 2). The solution was heated to reflux for 2 days. The crude compound was purified by chromatography on silica, employing EtOAc:petrol (1:1) as eluent, to give the title compound as a white solid (27%), m.p. >250° C. (decomp.).

### EXAMPLE 28

4-(6-Cyclohexylmethoxy-2-ylamino)-N,N-dimethylbenzenesulfonamide (NU 6128)

Prepared according to general procedure B using a mixture of n-BuOH and glycerol as the solvents and 4-amino-N,N-dimethylbenzenesulfonamide. The solution was heated to reflux for 3 days. The crude compound was purified by chromatography on silica (1% MeOH/99% DCM up to 4% MeOH/96% DCM as eluents) to afford the title compound as a brown solid (12%), m.p. 151-153° C.

### EXAMPLE 29

4-(6Cyclohexylmethoxy-2-ylamino)-N-ethylbenzenesulfonamide (NU6129)

Prepared according to general procedure B using n-BuOH and 4-amino-N-ethylbenzenesulfonamide. The solution was heated to reflux for 3 days. The crude compound was purified by chromatography on silica (40% EtOAc/60% petrol up to 100% EtOAc as eluents) to give the title compound as a white solid (45%), m.p. 245-247° C.

### EXAMPLE 30

4-(6Cyclohexylmethoxy-2-ylamino)-N-isobutylbenzenesulfonamide (NU6130)

Prepared according to general procedure B using n-BuOH and 4-amino-N-isobutylbenzenesulfonamide. The solution was heated to reflux for 3 days. The crude compound was purified by chromatography on silica (50% EtOAc/50% petrol up to 100% EtOAc as eluent) to afford the title compound as a white solid (17%), m.p. 240-242° C.

### EXAMPLE 31

4-(6-Cyclohexylmethoxy-9H-purin-2-ylamino)-N-cyclopropylbenzenesulfonamide (NU6131)

Prepared according to general procedure B using n-BuOH and 4-amino-N-cyclopropylbenzene-sulfonamide. The solution was heated to reflux for 2 days. The crude compound (oil) was purified by chromatography on silica (50% EtOAc/50% petrol up to 100% EtOAc as eluent) to lead to the title compound as a white solid (38%), m.p. 220-221° C.

### EXAMPLE 31

N-(3-Chloro-phenyl)-4-(6-cyclohexylmethoxy-9H-purin-2-ylamino)benzenesulfonamide (NU6132)

Prepared according to general procedure B using n-BuOH and 4-amino-N-(3-chlorophenyl)benzenesulfonamide. The solution was heated to reflux for 2 days. The crude compound was purified by chromatography on silica (50% EtOAc/50% petrol up to 100% EtOAc as eluent) to furnish the title compound as a white solid (16%), m.p. 248-249° C.

### EXAMPLE 32

4-(6-Cyclohexylmethoxy-9H-purin-2-ylamino)-N-propylbenzenesulfonamide (NU6133)

Prepared according to general procedure B using n-BuOH and 4-amino-N-propylbenzenesulfonamide. The solution was heated to reflux for 2 days. The crude compound was purified by chromatography on silica (50% EtOAc/50% petrol up to 100% EtOAc as eluent) to give the title compound as a white solid (32%), m.p. 207-208° C.

### EXAMPLE 33

4-(6-Cyclohexylmethoxy-9H-purin-2-ylamino)-N-(tetrahydrofuran-2-ylmethyl)benzenesulfonamide (NU6134)

Prepared according to general procedure B using n-BuOH and 4-amino-N-(tetrahydrofuran-2-ylmethyl)benzenesulfonamide. The solution was heated to reflux for 2 days. The crude compound was purified by chromatography on silica (50% EtOAc/50% petrol up to 100% EtOAc as eluent) to afford the title compound as a pale yellow solid (32%), m.p. 134-136° C.

### EXAMPLE 34

4-(6-Cyclohexylmethoxy-9H-purin-2-ylamino)-N-(2-methoxyethyl)benzenesulfonamide (NU6135)

Prepared according to general procedure B using n-BuOH and 4-amino-N-(2-methoxyethyl)benzenesulfonamide. The solution was heated to reflux for 2 days. The crude compound was purified by chromatography on silica (50% EtOAc/50% petrol up to 100% EtOAc as eluent) to give the title compound as a pale yellow solid (16%), m.p. 120-122° C.

### EXAMPLE 35

4-(6-Cyclohexylmethoxy-9H-purin-2-ylamino)-N,N-dimethylbenzamide (NU6136)

Prepared according to general procedure B using n-BuOH and 4-amino-N,N-dimethylbenzamide (from Example 3). The solution was heated to reflux for 2 days. The crude compound was purified by chromatography on silica (40% EtOAc/60% petrol up to 100% EtOAc as eluent) to give the title compound as a beige solid (35%), m.p. 184-186° C.

### EXAMPLE 36

4-(6-Cyclohexylmethoxy-9H-purin-2-ylamino)benzamide (NU6141)

Prepared according to general procedure B using n-BuOH and 4-aminobenzamide. The solution was heated to reflux for 16 h. The crude product was washed with a mixture of Et₂O/petrol and recrystallised from Et₂O/EtOH to give the title compound as a white solid (46%), m.p. 156-158° C.

### EXAMPLE 37

4-(6-Cyclohexylmethoxy-9H-purin-2-ylamino)-N-propylbenzamide (NU6142)

Prepared according to general procedure B using n-BuOH and 4-amino-N-propylbenzamide. The solution was heated to reflux for 2 days. The crude compound was purified by chromatography on silica (50% EtOAc/50% petrol up to 100% EtOAc as eluent) to afford the title compound as a white solid (50%), m.p. 226-228° C.

### EXAMPLE 38

[3-(6-Cyclohexylmethoxy-9H-purin-2-ylamino)phenyl]methanol (NU6151)

Prepared according to general procedure B using a mixture of n-BuOH/glycerol and (3-amino-phenyl)methanol. The solution was heated to reflux for 2 days. After cooling, the solvent was removed in vacuo. Water was added and the mixture was extracted with EtOAc, dried (MgSO₄), and the combined organic layers were evaporated, of the organic layer in vacuo, the crude compound was purified by trituration with Et₂O to lead after filtration to the title compound as a white solid (59%).

### EXAMPLE 39

4-(6-Cyclohexylmethoxy-9H-purin-2-ylamino)-(N-pyridin-2-yl)benzenesulfonamide (NU6154)

Prepared according to general procedure B using a mixture of n-BuOH/glycerol and 4-amino-N-pyridin-2-ylbenzenesulfonamide. The solution was heated under reflux for 1 day, cooled, and the solvent was removed in vacuo. Water was added and the product was extracted with EtOAc and dried (MgSO₄). After evaporation of the combined organic layers in vacuo, the crude product was purified by chromatography on silica (70% EtOAc/30% petrol up to 100% EtOAc as eluent) to give the title compound as a beige solid (32%).

### EXAMPLE 40

4-(6-Cyclohexylmethoxy-9H-purin-2-ylamino)-N-methylbenzamide (NU6139)

Prepared according to general procedure C using 4-amino-N-methyl-benzamide. The solution was heated to reflux for 1 day. The crude product was purified by chromatography (50% EtOAc/50% petrol up to 10% MeOH/90% DCM as eluent) to give the title compound as a pink solid (29%), m.p. 139-142° C.

### EXAMPLE 41

4-(6-Cyclohexylmethoxy-9H-purin-2-ylamino)-N,N-diethylbenzamide (NU6140)

Prepared according to general procedure C using 4-amino-N,N-diethylbenzamide. The solution was heated to reflux for 1 day. The crude product was purified by chromatography (50% EtOAc/50% petrol up to 100% EtOAc as eluent) to afford the title compound as a pale brown solid (29%), m.p. 138-140° C.

### EXAMPLE 42

4-(6-Cyclohexylmethoxy-9H-purin-2-ylamino)-N-ethylbenzamide (NU6143)

Prepared according to general procedure C using a mixture of n-BuOH/glycerol and 4-amino-N-ethylbenzamide. The solution was heated to reflux for 3 days. The crude product was purified by chromatography (50% EtOAc/50% petrol up to 100% EtOAc as eluent) to afford the title compound as a yellow solid (11%), m.p. 156-158° C.

### EXAMPLE 43

4-(6-Cyclohexylmethoxy-9H-purin-2-ylamino)-N-(2-hydroxyethyl)benzenesulfonamide (NU6144)

Prepared according to general procedure C using a mixture of n-BuOH/glycerol and 4-amino-N-(2-hydroxyethyl)benzenesulfonamide. The solution was heated to reflux for 3 days. The crude product was purified by chromatography (5% MeOH/95% DCM as eluent) to give the title compound as a beige solid (8%), m.p. 158-160° C.

### EXAMPLE 44

4-(6-Cyclohexylmethoxy-9H-purin-2-ylamino)-N-thiazol-2-ylbenzenesulfonamide (NU6145)

Prepared according to general procedure C using a mixture of n-BuOH/glycerol and 4-amino-N-thiazol-2-ylbenzenesulfonamide. The solution was heated to reflux for 3 days. The crude product was purified by chromatography (5% MeOH/95% DCM up to 10% MeOH/90% DCM as eluent) to give the title compound as a beige solid (4%), m.p. 152-154° C.

### EXAMPLE 45

4-(6-Cyclohexylmethoxy-9H-purin-2-ylamino)-N-(2-hydroxypropyl)benzenesulfonamide (NU6146)

Prepared according to general procedure C using a mixture of n-BuOH and glycerol, and 4-amino-N-(2-hydroxypropyl)benzenesulfonamide. The solution was heated to reflux for 3 days. After cooling, the solvent was removed in vacuo. The crude product was purified by chromatography (50% EtOAc/50% petrol up to 100% EtOAc as eluent) to furnish the title compound as a white solid (13%), m.p. 236-238° C.

### EXAMPLE 46

4-(6-Cyclohexylmethoxy-9H-purin-2-ylamino)-N-phenyl-benzenesulfonamide (NU6152)

Prepared according to general procedure C using a mixture of n-BuOH/glycerol and 4-amino-N-phenyl-benzenesulfonamide. The solution was heated to reflux for 3 days. The crude product was purified by chromatography (10% MeOH/90% DCM as eluent) to give the title compound as a yellow solid.

### EXAMPLE 47

4-(6-Cyclohexylmethoxy-9H-purin-2-ylamino)-N-(2,3-dihydroxypropyl)benzenesulfonamide (NU6153)

Prepared according to general procedure C using a mixture of n-BuOH/glycerol and 4-amino-N-(2,3-dihydroxy-propyl)-benzenesulfonamide. The solution was heated to reflux for 3 days. The product was purified by trituration with a mixture of Et₂O/petrol and EtOH to give the title compound as a white solid (11%).

### EXAMPLE 48

4-(6-Cyclohexylmethoxy-9H-purin-2-yl)-(4-methanesulfonylphenyl)amine (NU6155)

Prepared according to general procedure C using a mixture of n-BuOH/glycerol and 4-methanesulfonylphenylamine. The solution was heated to reflux for 3 days. The crude product was purified by chromatography (70% EtOAc/30% petrol followed by 5% MeOH/95% DCM as eluent) to furnish the title compound as a beige solid (4.5%).

### EXAMPLE 49

N-Benzyl-4-(cyclohexylmethoxypurine-2-ylamino)-benzenesulfonamide (NU6123)

Prepared according to general procedure D using a mixture of EtOH and glycerol, and 4-amino-N-benzylbenzenesulfonamide. The solution was heated to reflux for 3 days. Recrystallisation from MeOH gave the target compound as an off white solid (12%), m.p. 222-224° C.

### EXAMPLE 50

4-[(6-Cyclohexylmethoxypurine-2-ylamino)methyl]benzenesulfonamide (NU6124)

Prepared according to general procedure D using a mixture of n-BuOH and glycerol, and 4-aminomethylbenzenesulfonamide. The solution was heated to reflux for 2 days. The yellow product was washed with diethyl ether to furnish the title compound as a pale green solid (11%), m.p. 150° C. (decomp.).

### EXAMPLE 51

4-[6-(2-Methyl-cyclohexylmethoxy)-9H-purin-2-ylamino]benzenesulfonamide (NU6158)

Prepared according to general procedure B using O⁶-[2-(2-methyl-cyclohexyl)-ethoxy]-2-fluoropurine and sulfanilamide. The solution was heated to reflux for 2 days. The crude compound (oil) was purified by chromatography (60% EtOAc/40% petrol up to 3% MeOH97% DCM) to lead to the title compound as a pale yellow solid (15%). ¹H NMR (CD₃OD) δ1.1 (3H, d, J=7.5 Hz, —CH₃), 1.2-1.9 (10H, m, cyclohexyl), 4.5 (2H, d, J=7.5 Hz, —CH₂O—), 7.8 (2H, d, J=7.5 Hz, 2×Ar—H), 8.0 (2H, d, J=7.5 Hz, 2×Ar—H).

### EXAMPLE 52

4-(6-Cyclohexylmethoxy-9H-purin-2-ylamino)-N-(2,6dimethylpyrimidin-4-yl)-benzenesulfonamide (NU6160)

Prepared according to general procedure B using (2,6-dimethylpyrimidin-4-yl)-benzenesulfonamide. The solution was heated to reflux for 2 days. The crude compound (oil) was purified by chromatography (3% MeOH/97% DCM) and recrystallisation from MeOH to give the title compound as a pale yellow solid (9%). m.p. 233° C.; ¹H NMR (d⁶-DMSO) δ0.8-1.3 (5H, m, cyclohexyl), 1.5-1.9 (6H, m, cyclohexyl), 2.2 (3H, s, CH₃—), 2.3 (3H, s, CH₃—), 4.3 (2H, d, J=7.5 Hz, —CH₂O—), 6.7 (1H, m, Ar—H), 7.7 (2H, d, J=7.5 Hz, 2×Ar—H), 7.9 (2H, d, J=7.5 Hz, 2×Ar—H), 9.7 (1H, s, —NH), 12.9 (1H, s, —NH). MS M⁺=508 (100%).

### EXAMPLE 53

4-[6-Cyclohexylmethoxy-9H-purin-2-ylamino]-benzene(3′-methoxy)sulfonamide (NU6161)

Prepared according to general procedure B using 4-amino-2-methoxy-benzenesulfonamide. The solution was heated to reflux for 1 day. The crude compound was purified by chromatography (50% EtOAc/50% petrol up to 10% MeOH/90% DCM) to afford the title compound as a yellow solid (39%). M.p. 170-173° C. ¹H NMR (d⁶-DMSO) δ0.97 (5H, m, cyclohexyl), 1.61 (6H, m, cyclohexyl), 3.82 (3H, s, OCH₃), 4.26 (2H, d, OCH₂), 6.79 (2H, s, NH₂), 7.31 (1H, d, Ar.H), 7.50 (1H, d, Ar.H), 7.80 (1H, s, Ar.H), 8.01 (1H, s, H⁸), 9.52 (1H, s, N₉H exch in D₂O).

### EXAMPLE 54

4-[6-(Cyclohex-3-enylmethoxy)-9H-purin-2ylamino]-benzenesulfonamide (NU6162)

Prepared according to general procedure B using O⁶-(2-cyclohex-3-enyl-ethoxy)-2-fluoropurine and sulfanilamide. The solution was heated to reflux for 1 day. The crude compound (oil) was purified by chromatography (70% EtOAc/30% petrol up to 7% MeOH/93% DCM) to give the title compound as a pale brown solid (2%). m.p. 165° C.

### EXAMPLE 55

4-(6-Cyclohexylmethoxy-9H-purin-2-ylamino)-N-(2,2,2-trifluoroethyl)-benzenesulfonamide (NU6163)

Prepared according to general procedure B using 4-amino-N-(2,2,2-trifluoroethyl)-benzenesulfonamide. The solution was heated to reflux for 2 days. The crude compound (oil) was purified by chromatography (40°/EtOAc/60% petrol up to 10% MeOH/90% DCM), followed by recrystallisation from EtOAc-petrol to give the title compound as a pale beige powder (65%), m.p. 223.2-223.7° C. ¹H NMR (CDOD) δ8.04 (1H, s, Ar.H), 7.99 (2H, brs, Ar.H and H⁸), 7.88 (1H, s, Ar.H), 7.73 (1H, s, Ar.H), 4.39 (2H, d, OCH₂), 3.58 (2H, q, CH₂—CF₃), 2.01-1.78 (6H, m, cyclohexyl), 1.33-1.17 (4H, m, cyclohexyl), 0.88 (1H, m, cyclohexyl). MS M⁺484.12 (25%) (Calc. as 484.150445), [M-1] 483.11 (100%).

### EXAMPLE 56

2-[4-(6-Cyclohexylmethoxy-9H-purin-2-ylamino)-phenyl]acetamide (NU6164)

Prepared according to general procedure B using 2-(4-amino-phenyl)acetamide. The solution was heated to reflux for 1 day. The crude compound was recrystallised from ether-EtOAc to afford the title compound as a beige solid (6%), m.p. 233.5-233.8° C.; ¹H NMR (d₆-DMSO) δ7.93 (1H, s, H⁸), 7.65 (2H, d, Ar.H), 7.36 (1H, s, NH), 7.09 (2H, d, Ar.H), 6.80 (1H, s, NH), 4.28 (2H, d, OCH₂), 3.20 (2H, d, CH₂), 1.80-1.60 (6H, m, cyclohexyl), 1.20-1.08 (5H, m, cyclohexyl). MS M⁺380.22 (20%) (Calc. as 484.150445) [M-1] 379.19 (100%).

### EXAMPLE 57

4-[6-Cyclopentylmethoxy-9H-purin-2-ylamino)-benzenesulfonamide (NU6166)

The starting material for this reaction, namely O⁶-(cyclopentylmethoxy)-2-fluoropurine was prepared in exactly the same manner as the O⁶-(cyclohexylmethoxy)-2-fluoropurine described in Examples 7 and 8 with the exception that cyclopentylmethanol (4 equiv.) is used instead of the cyclohexylmethanol.

The compound NU6166 was then prepared according to general procedure B using O⁶-(cyclopentylmethoxy)-2-fluoropurine and sulfanilamide. The solution was heated to reflux for 1 day. The crude compound (oil) was purified by chromatography (70% EtOAc/30% petrol up to 7% MeOH/93% DCM) to give the title compound as a pale yellow solid (6%).

### EXAMPLE 58

4-(6-Cyclohexylmethoxy-9H-purin-2-ylamino)-N-methyl-benzamide (NU6167)

Prepared according to general procedure C using 4-amino-N-(2(S)-hydroxy-propyl)-benzenesulfonamide. The solution was heated to reflux for 1 day. The crude compound (oil) was purified by chromatography (50% EtOAc/50% petrol up to 10% MeOH/90% DCM) to furnish the title compound as a pink solid (29%). m.p. 139-142° C.; ¹H NMR (d⁶-DMSO) δ1.09 (5H, m, cyclohexyl), 1.89 (6H, m, cyclohexyl), 2.44 (3H, t, CH₃), 4.56 (2H, d, OCH₂), 7.83 (4H, q, Ar.H), 8.35 (1H, s, H⁸), 9.8 (1H, s, N⁹H). MS M⁺381.

TABLE 1
Inhibition of CDK1 and CDK2 by 2-anilinopurines

| | | | IC₅₀ ± SD | | | |
|---|---|---|---|---|---|---|
| Number | Name | Structure | CDK1 | | CDK2 | |
| Ref. | Olomoucine | 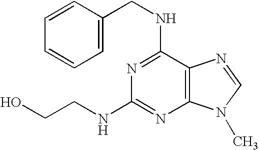 | 7.5 ± 0.1 | μM | 5.3 ± 0.5 | μM |
| NU2058 | 2-aniino-6-cyclohexyl- methyloxypurine C₁₂H₁₇N₅O MW = 247.30 | 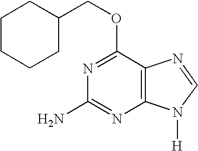 | 7 ± 0.7 | μM | 17 ± 2 | μM |
| NU6086 | 6-cyclohexylmethoxy-2-(3- chloroanilino)-purine C₁₈H₂₀ClN₅O MW = 357.14 | 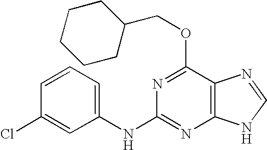 | 2.2 ± 0.4 | μM | 2.4 ± 0.3 | μM |
| NU6094 | 2-anilino-6-cyclohexyl- methoxypurine C₁₈H₂₁N₅O MW = 323.40 | 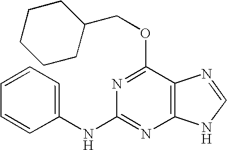 | 1.6 ± 0.1 | μM | 0.97 ± 0.03 | μM |
| NU6095 | 2-(3′-bromoanilino)-6- cyclohexylmethoxypurine C₁₈H₂₀BrN₅O MW = 402.29 | 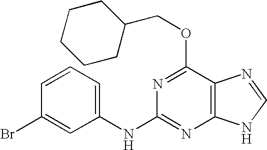 | 5.3 ± 0.8 | μM | 6.8 ± 4.8 | μM |
| NU6096 | 6-cyclohexylmethoxy-2-(3′- methoxyanilino)purine C₁₉H₂₃N₅O₂MW = 353.42 | 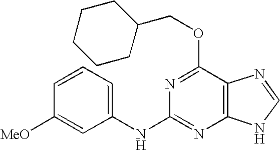 | 2.2 ± 0.2 | μM | 1.8 ± 0.2 | μM |
| NU6098 | 6-cyclohexylmethoxy-2-(3- fluoroanilino)purine C₁₈H₂₀FN₅O MW = 341.39 | 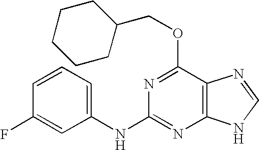 ˜10 | μM | ˜10 | μM | |
| NU6099 | 6-cyclohexylmethoxy-2-(3- ethylanilino)purine C₂₀H₂₅N₅O MW = 351.45 | 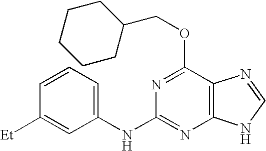 | >10 | μM | >10 | μM |
| NU6100 | 6-cyclohexylmethoxy-2-(4- methoxyanilino)purine C₁₉H₂₃N₅O₂MW = 353.42 | 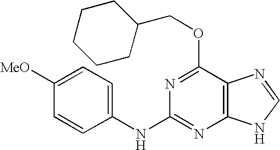 | 0.8 ± 0.01 | μM | 0.7 ± 0.03 | μM |
| NU6101 | 6-cyclohexylmethoxy-2-(3- methylmercaptoanilino)- purine C₁₉H₂₃N₅OS MW = 369.48 | 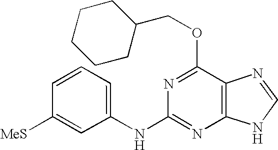 | 2.4 ± 0.2 | μM | 1.7 ± 0.1 | μM |
| NU6102 | 6-cyclohexylmethoxy-2-(4′- sulfamoylanilino)purine C₁₉H₂₃N₅OS MW = 369.48 | 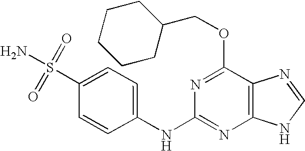 | 9.5 ± 0.1 | nM | 5.4 ± 1.0 | nM |
| NU6116 | 6-cyclohexylmethoxy-2-(4′- fluoroanilino)purine C₁₈H₂₀FN₅O MW = 341.39 | 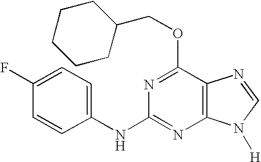 | >10 | μM | >10 | μM |
| NU6117 | 6-cyclohexylmethoxy-2-(4′- methylmercaptoanilino)- purine C₁₉H₂₃N₅OS MW = 369.48 | 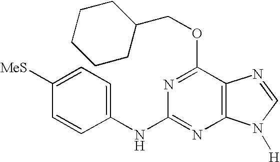 | >10 | μM | >10 | μM |
| NU6118 | methyl 4-N-(6-cyclohexyl- methoxypurin-2- yl)aminobenzoate C₂₀H₂₃N₅O₃MW = 381.43 | 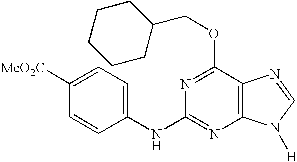 | ˜10 | μM | ˜10 | μM |
| NU6119 | 6-cyclohexylmethoxy-2-(3′- chloro-4′-fluoroanilino)- C₁₈H₁₉ClFN₅O MW = 375.83 | 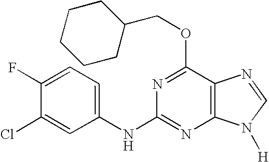 | ˜10 | μM | ˜10 | μM |
| NU6120 | 6-cyclobexylmethyloxy-2- (4′-hydroxyanilino)purine C₁₈H₂₁N₅O₂MW = 339.40 | 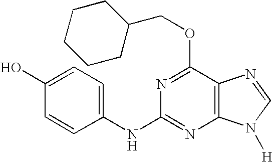 | 94 ± 12 | nM | 69 ± 1 | nM |
| NU6121 | 6-cyclohexylmethyloxy-2- (4′-cyanomethylanilino)- purine C₂₀H₂₂N₆O MW = 362.43 | 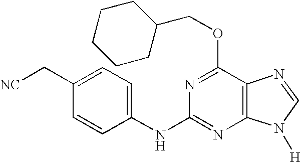 | 0.3 ± 0.1 | μM | 0.3 ± 0.03 | μM |
| NU6123 | N-Benzyl-4- (cyclohexylmethoxypurine- 2-ylamino)- benzenesulfonamide C₂₅H₂₈N₆O₃S MW = 492.60 | 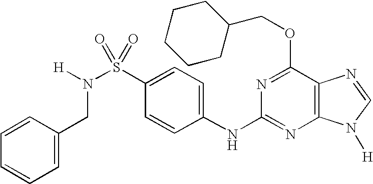 | 0.7 ± 0.3 | μM | 0.2 ± 0.1 | μM |
| NU6124 | 4-[(6-Cyclohexylmethoxy- purine-2-ylamino)methyl]- benzenesulfonamide C₁₉H₂₄N₆O₃S MW = 416.50 | 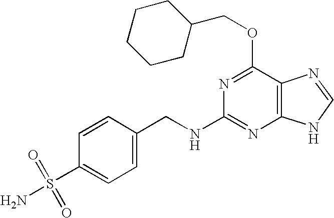 | 17 ± 0.3 | μM | 15, 13 | μM |
| NU6125 | 4-(6-Cyclohexyl-methoxy- purin-2-yl)amino- acetophenone C₂₀H₂₃N₅O₂MW = 365.43 | 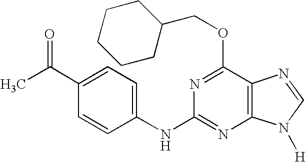 | 0.5 ± 0.2 | μM | 0.3 ± 0.04 | μM |
| NU6126 | Methyl-4-[(6′- cyclohexylmethoxy-purin- 2-yl)amino]phenylsulfoxide C₁₉H₂₃N₅O₂S MW = 385.48 | 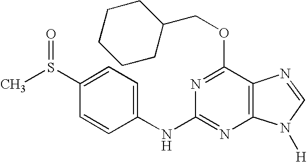 | 0.2 ± 0.1 | μM | 0.1 ± 0.01 | μM |
| NU6127 | 4-(6-Cyclohexylmethoxy-2- ylamino)-N-methyl- benzenesulfonamide C₁₉H₂₄N₆O₃S MW = 416.50 | 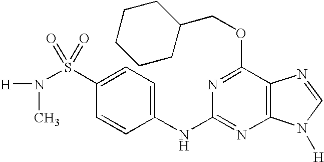 | 9, 10 | nM | 7 ± 0.1 | nM |
| NU6128 | 4-(6-Cyclohexylmethoxy-2- ylamino)-N,N-dimethyl- benzenesulfonamide C₂₀H₂₆N₆O₃S MW = 430.52 | 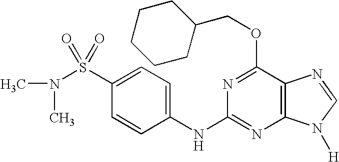 | 77 ± 5 | nM | 56 ± 20 | nM |
| NU6129 | 4-(6-Cyclohexylmethoxy-2- ylamino)-N-ethyl- benzenesulfonamide C₂₀H₂₆N₆O₃S MW = 430.52 | 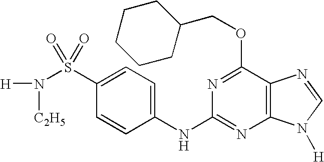 | 7, 9 | nM | 8 ± 2 | nM |
| NU6130 | 4-(6-Cyclohexylmethoxy-2- ylamino)-N-isobutyl- benzenesulfonamide C₂₂H₃₀N₆O₃S MW = 458.58 | 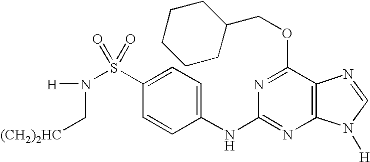 | 71 ± 9 | nM | 69 ± 13 | nM |
| NU6131 | 4-(6-Cyclohexylmethoxy- 9H-purin-2-ylamino)-N- cyclopropyl- benzenesulfonamide C₂₁H₂₆N₆O₃S MW = 442.54 | 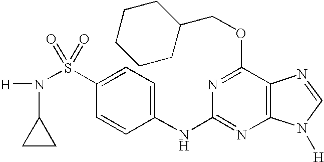 | 13 ± 5 | nM | 8 ± 0.3 | nM |
| NU6132 | N-(3-Chloro-phenyl)-4-(6- cyclohexylmethoxy-9H- purin-2-ylamino)- benzenesulfonamide C₂₁H₂₅N₆O₃SCl MW = 513.02 | 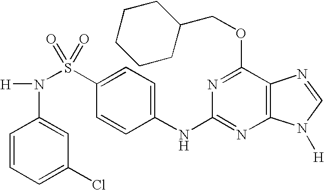 | 5.6 | μM | 5.7 | μM |
| NU6133 | 4-(6-Cyclohexylmethoxy- 9H-purin-2-ylainino)-N- propyl-benzenesulfonamide C₂₁H₂₈N₆O₃S MW = 444.55 | 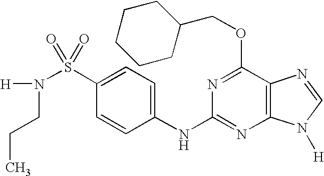 | 34 ± 4 | nM | 17 ± 13 | nM |
| NU6134 | 4-(6-Cyclobexylmethoxy- 9H-purin-2-ylamino)-N- (tetrahydro-furan-2- ylmethyl)-benzenesulfo- namide C₂₃H₃₀N₆O₄S MW = 486.59 | 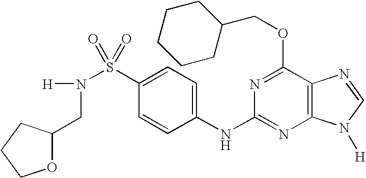 | 7.2, 7.3 | nM | 7 ± 3.2 | nM |
| NU6135 | 4-(6-Cyclohexylmethoxy- 9H-purin-2-ylamino)-N-(2- methoxy-ethyl)- benzenesulfonamide C₂₁H₂₈N₆O₄S MW = 460.55 | 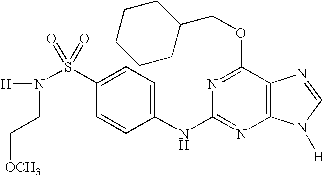 | 18 ± 4 | nM | 6 ± 1.5 | nM |
| NU6136 | 4-(6-Cyclohexylmethoxy- 9H-purin-2-ylamino)-N,N- dimethyl-benzamide C₂₁H₂₆N₆O₂MW = 394.48 | 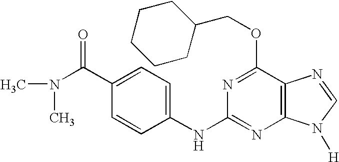 | 0.6 | μM | 0.2 | μM |
| NU6139 | 4-(6-Cyclohexylmethoxy- 9H-purin-2-ylamino)-N- methyl-benzamide C₂₀H₂₄N₆O₂MW = 380.45 | 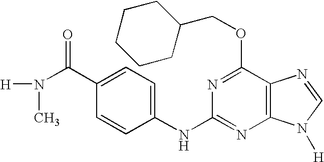 | 0.7 | μM | 0.3 | μM |
| NU6140 | 4-(6-Cyclohexylmethoxy- 9H-purin-2-ylamino)-N,N- diethyl-benzamide C₂₃H₃₀N₆O₂MW = 422.53 | 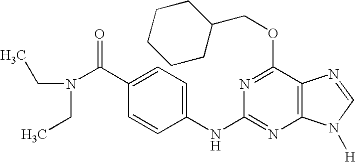 | 0.7 | μM | 0.5 | μM |
| NU6141 | 4-(6-Cyclohexylmethoxy- 9H-purin-2-ylamino)- benzamide C₁₉H₂₂N₆O₂MW = 366.43 | 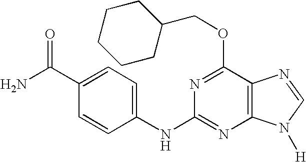 | 83 | nM | ˜100 | nM |
| NU6142 | 4-(6-Cyclohexylmethoxy- 9H-purin-2-ylamino)-N- propyl-benzamide C₂₂H₂₈N₆O₂MW = 408.50 | 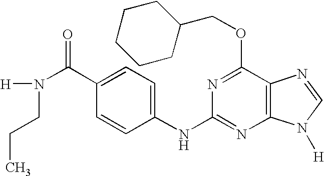 | 0.42 | μM | ˜100 | nM |
| NU6143 | 4-(6-Cyclohexylmethoxy- 9H-purin-2-ylamino)-N- ethyl-benzamide C₂₁H₂₆N₆O₂MW = 394.48 | 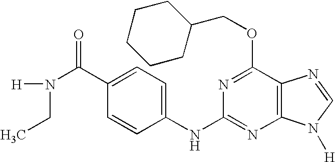 | >100 | nM | >100 | nM |
| NU6144 | 4-(6-Cyclohexylmethoxy- 9H-purin-2-ylamino)-N-(2- hydroxy-ethyl)- benzenesulfonamide C₂₀H₂₆N₆O₄S MW = 446.52 | 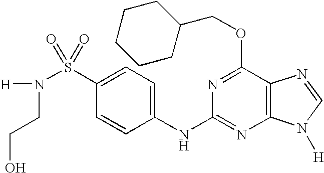 | 8 ± 0.2 | nM | 4 ± 1.5 | nM |
| NU6145 | 4-(6-Cyclohexylmethoxy- 9H-purin-2-ylamino)-N- thiazol-2-yl- benzenesulfonamide C₂₁H₂₃N₇O₃S₂MW = 485.58 | 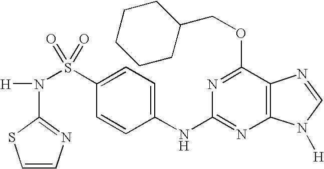 | 37 | nM | 24 | nM |
| NU6146 | 4-(6-Cyclohexylmethoxy- 9H-purin-2-ylamino)-N-(2- hydroxy-propyl)- benzenesulfonamide C₂₁H₂₈N₆O₄S MW = 460.55 | 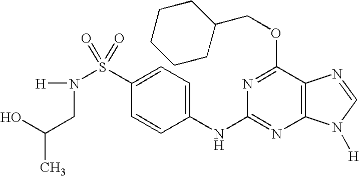 | 14 ± 7 | nM | 5 ± 1 | nM |
| NU6147 | 4-(6-Cyclohexylmethoxy- 9H-purin-2-ylamino)- benzoic acid C₁₉H₂₁N₅O₃MW = 367.41 | 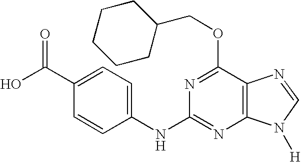 | 0.24 | μM | — | |
| NU6151 | [3-(6-Cyclohexylmethoxy- 9H-purin-2-ylamino)- phenyl]methanol C₁₉H₂₃N₅O₂MW = 353.42 | 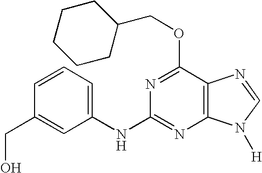 | 0.3 ± 0.01 | μM | 0.4 ± 0.1 | μM |
| NU6152 | 4-(6-Cyclohexylmethoxy- 9H-purin-2-ylamino)-N- phenylbenzene-sulfonamide C₂₄H₂₆N₆O₃S MW = 478.57 | 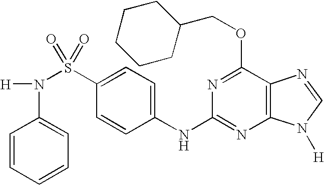 | 0.4 ± 0.1 | μM | 0.2 ± 0.02 | μM |
| NU6153 | 4-(6-Cyclohexylmethoxy- 9H-purin-2-ylamino)-N- (2,3-dihydroxypropyl)- benzenesulfonamide C₂₁H₂₈N₆O₅S MW = 476.55 | 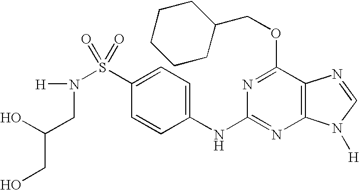 | 7 ± 1 | nM | 5 ± 2 | nM |
| NU6154 | 4-(6-Cyclohexylmethoxy- 9H-purin-2-ylamino)-(N- C₂₃H₂₅N₇O₃S MW = 479.56 | 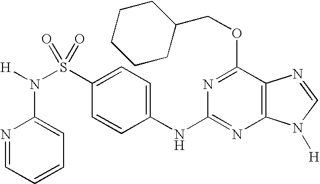 | 20 ± 6 | nM | 27 ± 2 | nM |
| NU6155 | 4-(6-Cyclohexylmethoxy- 9H-purin-2-yl)-(4-methane- sulfonylphenyl)amine C₁₉H₂₃N₅O₃S MW = 401.48 | 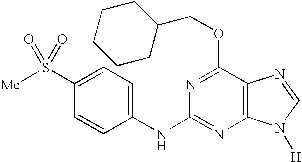 | 80 ± 5 | nM | 63 ± 7 | nM |
| NU6158 | 4-[6-(2-Methylcyclohexyl- methoxy)-9H-purin-2- ylamino]benzene- sulfonamide. C₁₉H₂₄N₆O₃S MW = 416.50 | 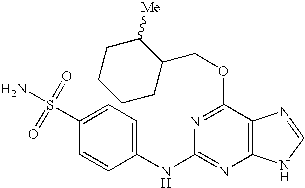 | 6 ± 1 | nM | 4 ± 0.3 | nM |
| NU6160 | 4-(6-Cyclohexylmethoxy- 9H-purin-2-ylamino)-N- (2,6-dimethylpyrimidin-4- yl)benzenesulfonamide C₂₄H₂₈N₈O₃S MW = 508.60 | 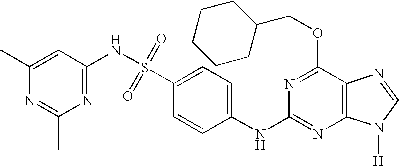 | 28 ± 2 | nM | 33 ± 6 | nM |
| NU6161 | 4[6-cyclohexylmethoxy- 9H-purin-2-ylamino)- benzene(3′-methoxy)- sulfonamide C₁₉H₂₄N₆SO₄MW = 432.50 | 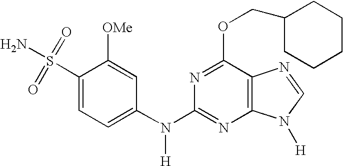 | 103 ± 4 | nM | 70 ± 8 | nM |
| NU6162 | 4-[6-(Cyclohex-3-enyl- methoxy)9H-purin-2- ylamino]-benzene- sulfonamide C₁₈H₂₀N₆O₃S MW = 400.46 | 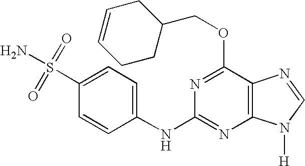 | 21 ± 4 | nM | 10 ± 1 | nM |
| NU6163 | 4-(6-Cyclohexylmethoxy- 9H-purin-2-ylamino)-N- (2,2,2-trifluoro-ethyl)- C₂₀H₂₃F₃N₆O₃S MW = 484.5 | 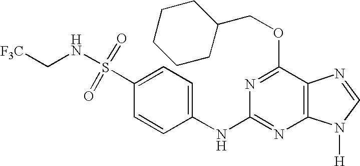 | 66 ± 5 | nM | 51 ± 16 | nM |
| NU6164 | 2-[4-(6-Cyclohexyl- methoxy-9H-pnrin-2- ylamino)phenylacetamide C₂₀H₂₄N₆O₂MW = 380.45 | 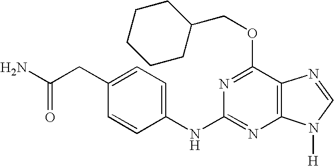 | 136 ± 22 | nM | 132 ± 20 | nM |
| NU6166 | 4-[6-Cyclopentyl-methoxy- 9H-purin-2-ylamino)- benzenesulfonamide C₁₇H₂₀N₆O₃S MW = 388.44 | 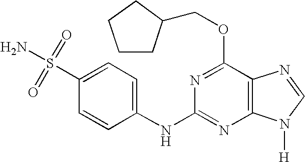 | 14 ± 3 | nM | 7 ± 1 | nM |
| NU6167 | 4-(6-Cyclohexylmethoxy- 9H-purin-2-ylamino)-N-(2- hydroxy-propyl)benzene- sulfonamide C₂₁H₂₈N₆SO₄MW = 460.55 | 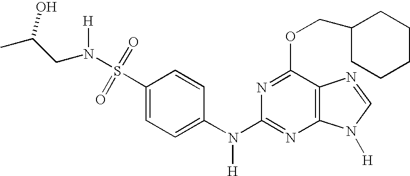 | 9 ± 0.2 | nM | 6 ± 1 | nM |

1. A purine compound having CDK-inhibiting activity useful for the treatment of tumours and other cell proliferation disorders in mammals, said compound having the structural formula I below: 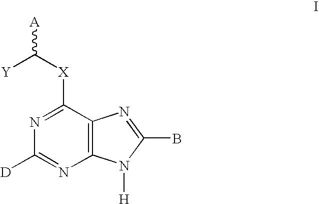
or a pharmaceutically acceptable salt and/or prodrug form thereof, wherein:
X is O, S or CHRₓ
where Rₓ is H or C₁₋₄ alkyl;
D is NZ₁Z₂
where Z₁ is selected from H, C₁₋₄ alkyl, C₁₋₄ hydroxyalkyl, an unsubstituted or substituted aryl or heteroaryl, and an unsubstituted or substituted aralkyl or heteroaralkyl group, and Z₂ is selected from an unsubstituted or substituted aryl or heteroaryl, and an unsubstituted or substituted aralkyl or heteroaralkyl group;
A is selected from H, C₁₋₄ alkyl, C₁₋₄ alkoxy, hydroxy, CH₂(CH₂)ₙOH (n=1-4), and NRₐ₁Rₐ₂ where Rₐ₁ and Rₐ₂ are each independently H or C₁₋₄ alkyl;
B is selected from H, halo, C₁₋₄ alkyl, C₁₋₄ alkoxy, CF₃, an unsubstituted or substituted aryl or an unsubstituted or substituted aralkyl, and a hydroxy group that may undergo a C═O tautomeric rearrangement; and
Y comprises an unsubstituted or substituted 4- to 8-membered carbocyclic or heterocyclic ring, optionally forming part of a larger fused ring structure, or consists of an unsubstituted or substituted linear or branched hydrocarbon chain.

2. A purine compound as claimed in claim 1, wherein Z₁ is H.

3. A purine compound as claimed in claim 2, wherein Z₂ is an unsubstituted aryl or a substituted aryl having at least one substituent X′ selected from the following:
a C₁-C₆ linear or branched alkyl chain optionally substituted with one or more substituents selected from amino, halo, cyano, OH or a C₁-C₄ alkoxy; an unsubstituted or substituted aryl or aralkyl; halo; OH; SH; C₁-C₆ acyl; OR¹; SR¹; NR²R³; (CH₂)_{q}R²R³; NO₂; N₃; CN; O(CH₂)_{q}O in which the (CH₂)_{q} moiety is optionally substituted by one or more functional groups; COR⁴; CONR⁵R⁶; (CH₂)_{q}CONR⁵R⁶; COOR⁴; SOR⁴; SO₂R⁴ and SO₂NR⁵R⁶;
wherein R¹, R² and R³ each represent independently a C₁-C₆ linear or branched alkyl which is optionally substituted with one or more substituents selected from amino, OH, halo or a C₁-C₄ alkoxy group; and R⁴, R⁵ and R⁶ represent independently H; a C₁-C₆ linear or branched alkyl optionally substituted with one or more substituents selected from halo, OH, C₁₋₄ alkoxy or NR⁷R⁸; a C₃-C₈ cycloalkyl; or an unsubstituted or substituted aralkyl, aryl or a group that comprises an unsubstituted or substituted 5- to 7-membered heterocyclic ring; and where q is in the range 1 to 6 and R⁷ and R⁸ represent independently H, a C₁-C₆ linear or branched alkyl chain optionally substituted with OH, amino or substituted amino.

4. A purine compound according to claim 3, wherein said compound is a substituted aryl comprising one to three substituents X′.

5. A purine compound as claimed in claim 3, wherein Z₂ is substituted or unsubstituted phenyl.

6. A purine compound as claimed in claim 1 or 2, wherein said compound has the structural formula IV shown below: 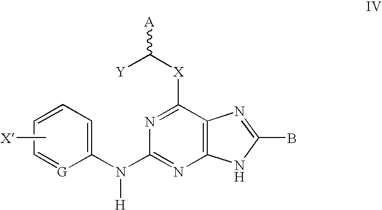
and wherein X′ is H, or a substituent selected from a C₁-C₆ linear or branched alkyl chain optionally substituted with one or more substituents selected from amino, halo, cyano, OH or a C₁-C₄ alkoxy; an unsubstituted or substituted aryl or aralkyl; halo; OH; SH; C₁-C₆ acyl; OR¹; SR¹; NR²R³; (CH₂)_{q}R²R³; NO₂; N₃; CN; O(CH₂)_{q}O in which the (CH₂)_{q} moiety is optionally substituted by one or more functional groups; COR⁴; CONR⁵R⁶; (CH₂)_{q}CONR⁵R⁶; COOR⁴; SOR⁴; SO₂R⁴ and SO₂NR⁵R⁶;
where R¹, R² and R³ each represent independently a C₁-C₆ linear or branched alkyl which is optionally substituted with one or more substituents selected from amino, OH, halo or a C₁-C₄ alkoxy group; and R⁴, R⁵ and R⁶ represent independently H; a C₁-C₆ linear or branched alkyl optionally substituted with one or more substituents selected from halo, OH, C₁₋₄ alkoxy or NR⁷R⁸; C₃-C₈ cycloalkyl; or an unsubstituted or substituted aralkyl, aryl or a group that comprises an unsubstituted or substituted 5- to 7-membered heterocyclic ring; and where q is in the range 1 to 6 and R⁷ and R⁸ represent independently H, a C₁-C₆ linear or branched alkyl chain optionally substituted with OH, amino or substituted amino; and G is CH or N.

7. A purine compound as claimed in claim 6, wherein G is CH.

8. A purine compound as claimed in claim 6 or 7, wherein at least one substituent X′ is present in the 4′ or para position.

9. A purine compound as claimed in claim 3 to 8, wherein any one of X′, R¹, R², R³, R⁴, R⁵, or R⁶ represent a C₁-C₆ alkyl group substituted with three halo substituents.

10. A purine compound as claimed in claim 9, wherein said alkyl group substituted with three halo substituents is selected from CF₃ or CH₂CF₃.

11. A purine compound as claimed in any one of claims 3 to 8, wherein R⁴, R⁵ or R⁶ represent independently a group comprising an unsubstituted or substituted heterocyclic ring having one or more heteroatoms selected from N, O or S.

12. A purine compound as claimed in claim 11, wherein said heterocyclic ring is optionally substituted with one or more substituents selected from C₁-C₄ alkyl, C₁-C₄ alkoxy, halo, OH and amino.

13. A purine compound as claimed in claim 11 or 12, wherein said group is a heteroaryl or heteroaralkyl group.

14. A purine compound as claimed in claim 3 or 6, wherein R⁵ and R⁶ are linked to form a 5- to 7-membered heterocyclic ring.

15. A purine compound as claimed in claim 3 or 6, wherein R⁷ or R⁸ are linked to form a 5- to 7-membered heterocyclic ring.

16. A purine compound as claimed in claim 14 or 15, wherein said heterocyclic ring comprises one or more additional heteroatoms selected from N, O or S.

17. A purine compound as claimed in claim 14 or 15 wherein said heterocyclic ring is a piperidino or a morpholino ring structure.

18. A purine compound as claimed in any one of claim 3 to 7, wherein two substituents X′ are present on adjacent carbon atoms and are linked to form a cyclic substituent.

19. A purine compound as claimed in claim 18, wherein Z₂ has the structural formula II shown below 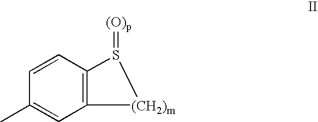
where p=1 or 2 and m is in the range of 2 to 5.

20. A purine compound as claimed in claim 18, wherein Z₂ has the structural formula III shown below 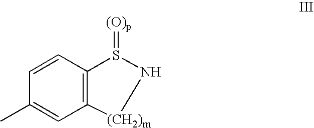
where p=1 or 2 and m is in the range of 2 to 5.

21. A purine compound as claimed in any preceding claim, wherein Y comprises a ring structure that includes polar hydroxyl substituents.

22. A purine compound as claimed in any preceding claim, wherein Y is a cycloalkane or cycloalkene ring.

23. A purine compound as claimed in any preceding claim in which Y is a 5- or 6-membered cycloalkane or cycloalkene ring having one or two double bonds.

24. A purine compound as claimed in claim 22 or 23 except that one or two of the carbon atoms in the cycloalkane or cycloalkene ring are replaced by hetero atoms or groups.

25. A purine compound as claimed in claim 24, wherein said hetero atoms or groups are selected from O, S, NR′ (where R′ is H or C₁₋₄ alkyl) and, in a cycloalkene ring, —N═.

26. A purine compound as claimed in claim 1, wherein Y is a substituted 4- to 8-membered carbocyclic or heterocyclic ring wherein the or each substituent is selected from H, C₁₋₄ alkyl, OH, C₁₋₄ alkoxy, halogen, CF₃, CN, N₃ and NR_{y1}R_{y2} where R_{y1} and R_{y2} are each independently H or C₁₋₄ alkyl.

27. A purine compound as claimed in claim 26, wherein two of the said substituents are on adjacent atoms of the ring and are linked to form an additional fused carbocyclic or heterocyclic ring structure.

28. A purine compound as claimed in claim 1 in which Y comprises a ring structure represented by one of the following structural formulae: 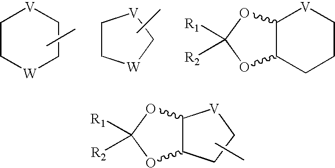
where V and W are each selected independently from
O, S, NR′ (R′ is H or C₁₋₄ alkyl)
and CH₂ or ═CH—; and
R₁ and R₂ are each H or C₁₋₄ alkyl.

29. A purine compound as claimed in any preceding claim, wherein A is H, B is H and X is O.

30. A purine compound as claimed in any preceding claim, wherein said purine compound is further characterised by one of the following features:
(i) aryl substituents, when present, are phenyl;
(ii) aralkyl substituents, when present, are benzyl;
(iii) halo substituents, when present, are selected from fluorine, chlorine, bromine and iodine atoms.

31. A purine compound having a structural formula as shown below:

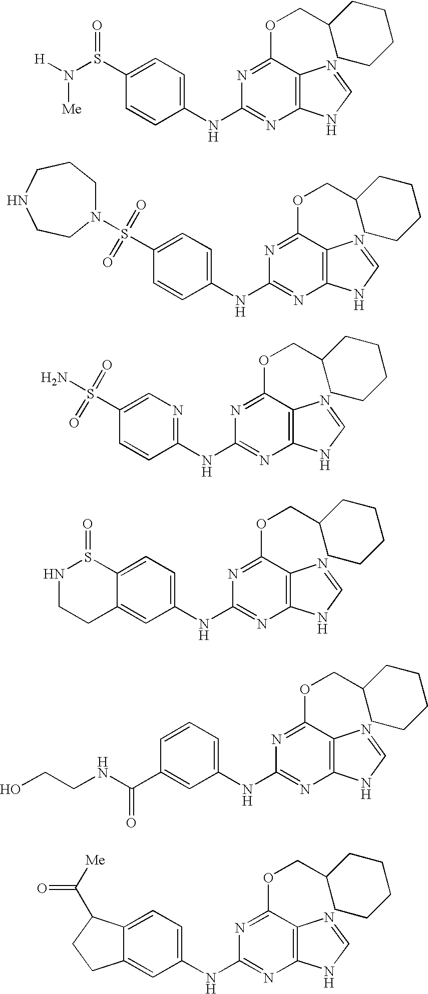

32. A purine compound wherein said compound in one of the following:
2-Anilino-6-cyclohexylmethoxypurine
2-(3′-Bromoanilino)-6-cyclohexylmethoxypurine
6-Cyclohexylmethoxy-2-(3′-chloroanilino)purine
6-Cyclohexylmethoxy-2-(4′-fluoroanilino)purine
6-Cyclohexylmethoxy-2-(3′-fluoroanilino)purine
6-Cyclohexylmethoxy-2-(3′-ethylanilino)purine
6-Cyclohexylmethoxy-2-(4′-methoxyanilino)purine
6-Cyclohexylmethoxy-2-(3′-methoxyanilino)purine
6-Cyclohexylmethoxy-2-(3′-methylmercaptoanilino)purine
6-Cyclohexylmethoxy-2-(4′-methylmercaptoanilino)purine
Methyl 4-N-(6-cyclohexyl-methoxypurin-2-yl)aminobenzoate
6-Cyclohexylmethoxy-2-(3′-chloro-4′-fluoroanilino)purine
6-Cyclohexylmethyoxy-2-(4′-hydroxyanilino)purine
4-(6-Cyclohexyl-methoxylpurin-2-yl)amino-acetophenone
6-Cyclohexylmethoxy-2-(4′-sulfamoylanilino)purine
N-Benzyl-4-(cyclohexylmethoxypurine-2-ylamino)-benzenesulfonamide
4-[(6-Cyclohexylmethoxy-purine-2-ylamino)methyl]-benzenesulfonamide
Methyl-4-[(6′-cyclohexylmethoxylpurin-2-yl)amino]phenylsulfoxide
4-(6-Cyclohexylmethoxy-purin-2-ylamino)-N-methyl-benzenesulfonamide
4-(6-Cyclohexylmethoxy-purin-2-ylamino)-N,N-dimethylbenzene-sulfonamide
4-(6-Cyclohexylmethoxypurin-2-ylamino)-N-ethyl-benzenesulfonamide
4-(6-Cyclohexylmethoxypurin-2-ylamino)-N-isobutyl-benzenesulfonamide
4-(6-Cyclohexylmethoxy-9H-purin-2-ylamino)-N-cyclopropyl-benzenesulfonamide
N-(3-Chlorophenyl)-4-6-cyclohexylmethoxy-9H-purin-2-ylamino)-benzenesulfonamide
4-(6-Cyclohexylmethoxy-9H-purin-2-ylamino)-N-propyl-benzenesulfonamide
4-(6-Cyclohexylmethoxy-9H-purin-2-ylamino)-N-tetrahydrofuran-2-ylmethyl)-benzenesulfonamide
4-(6-Cyclohexylmethoxy-9H-purin-2-ylamino)-N-(2-methoxy-ethyl)-benzenesulfonamide
4-(6-Cyclohexylmethoxy-9H-purin-2-ylamino)-N,N-dimethyl-benzamide
4-(6-Cyclohexylmethoxy-9H-purin-2-ylamino)-N-methylbenzamide
4-(6-Cyclohexylmethoxy-9H-purin-2-ylamino)-N,N-diethyl-benzamide
4-(6-Cyclohexylmethoxy-9H-purin-2-ylamino)-benzamide
4(6-Cyclohexylmethoxy-9H-purin-2-ylamino)-N-propyl-benzamide
4-(6-Cyclohexylmethoxy-9H-purin-2-ylamino)-N-ethyl-benzamide
4-(6-Cyclohexyloxymethoxy-9H-purin-2-ylamino)-N-(2-hydroxy-ethyl)-benzenesulfonamide
4-(6-Cyclohexyloxymethoxy-9H-purin-2-ylamino)-N-thiazol-2-yl-benzenesulfonamide
4-(6-Cyclohexyloxymethoxy-9H-purin-2-ylamino)-N-(2-hydroxy-propyl)-benzenesulfonamide
4-(6-Cyclohexyloxymethoxy-9H-purin-2-ylamino)-benzoic acid
[3-(6-Cyclohexyloxymethoxy-9H-purin-2-ylamino)-phenyl]methanol
4-(6-Cyclohexylmethoxy-9H-purin-2-ylamino)-N-phenylbenzene-sulfonamide
4-(6-Cyclohexylmethoxy-9H-purin-2-ylamino)-N-(2,3-dihydroxypropyl)-benzenesulfonamide
4-(6-Cyclohexylmethoxy-9H-purin-2-ylamino)-(N-pyridin-2-yl)-benzenesulfonamide
4-(6-Cyclohexylmethoxy-9H-purin-2-yl)-(4-methane-sulfonylphenyl)amine
4-(6-Cyclohexylmethoxy-9H-purin-2-ylamino)-N-(3-diethylaminopropyl)-benzenesulfonamide
4-[6-(-2-Methyl-cyclohexylmethoxy)-9H-purin-2-ylamino]benzenesulfonamide
4-(6-Cyclohexylmethoxy-9H-purin-2-ylamino)-N-(2,6-dimethylpyrimidin-4-yl)-benzenesulfonamide
4-[6-Cyclohexylmethoxy-9H-purin-2-ylamino]-benzene(3′-methoxy)sulfonamide
4-[6-(Cyclohex-3-enylmethoxy)-9H-purin-2ylamino]-benzenesulfonamide
4-(6-Cyclohexylmethoxy-9H-purin-2-ylamino)-N-(2,2,2-trifluoroethyl)benzenesulfonamide
2-[4-(6-Cyclohexylmethoxy-9H-purin-2-ylamino)]-phenylacetamide
4-[6-Cyclopentylmethoxy-9H-purin-2-ylamino)-benzenesulfonamide
4-(6-Cyclohexylmethoxy-9H-purin-2-ylamino)-N-(2-hydroxypropyl) benzenesulfonamide

33. A purine compound as claimed in claims 1 to 32 for use in therapy.

34. A purine compound as claimed in claims 1 to 32 for use as an active pharmaceutical substance for the treatment of tumours or other cell proliferation disorders.

35. Use of a purine compound as claimed in claims 1 to 32 in the manufacture of a medicament for the treatment of tumours or other cell proliferation disorders.

36. A pharmaceutical formulation or composition comprising an effective CDK-inhibiting amount of a purine compound as claimed in claims 1 to 32 in combination with a pharmaceutically acceptable carrier.

37. A pharmaceutical formulation or composition as claimed in claim 36, wherein said formulation or composition is adapted for parenteral administration.

38. A unit dose pharmaceutical formulation or composition comprising an effective CDK-inhibiting amount of a purine compound as claimed in claims 1 to 32 in combination with a pharmaceutically acceptable carrier.

39. A pharmaceutical formulation or composition as claimed in any one of claims 36 to 38 for use in the treatment of tumours or other cell proliferation disorders.

40. A method of manufacturing a pharmaceutical formulation or composition for use in the treatment of tumours or other cell proliferation disorders, said method comprising the steps of mixing a purine compound as claimed in any one of claims 1 to 32 with a pharmaceutically acceptable additive, carrier, diluent or excipient.

41. A method of treatment which comprises administering to a mammal in need of such treatment a therapeutically effective or CDK-inhibiting amount of a purine compound as claimed in any one of claims 1 to 32 or a pharmaceutically acceptable salt or prodrug form thereof.
